SEQUENCE LISTING (91 sequences)

SEQ ID NO: 1 - DNA, 5751 bp, Salmonella pneumoniae
cggctacctgcagagataaactacctaacacgagagtaagtagtctgtctgtagtcgtccagtctgatgatttttacgatacttcgtttttctgatcatacagttgcctctcggcataggtcagaactttaccttgtctgggtggttcccgttggtcgtagatagattggatatcgctaggagaatccttttgaactggaggaaagagggcatcgatcaagctactgaatactttaactaagtcaaatatagtatttttcttagtagacctaacccttttttcataatttctaatggtgtttttacttatacctatcttagtacccaattcttattgagtccaaccattactagtctatattgttttatagttgattgagtttggaatagtacgctgtagctgctaaaacatttctagaaattaatttgactttcctaatagagttgttcatatcttatttcaatttactatagtacaaaactagaaaaggaaaaaatcatgaccaaaacaattcttgttacaggtgggactagctacattggctcccataccgttaaagctcttttaaatgctggctatcaggtgcatgtcctcgataatctctccacaggaaatcgagcggctgtggatagtcgtgctagctttaaagaactggatgtttatgatgctagcgccttaaaggcttacttagaagaaaatcagattgatgctgtcctccattgtgcaggtgaaattgttgtgagcgaaagtattgaaaatccaagtaaatacttcactgccaacgttgctggtatgaaccaagttctcaaagtcttatctgaagttggcattcaaaaaatcatgttctcttcgactgcttctctctatggaaataactgtattgacaagccggtgactgaagataccctgctcgaccctgtcaacccttatgcagagacaaaactgatgggcgaacgaatgatttactggatggccaatcgctacgactggaaatatgttattttccgttactttaatgttgctggggctgaaatggatgcttcaaacggtctgcgtgtgaaaaatccaactcacatcattccaaatatcaacaagaccgcattgggacaaaatgatagcttaaaaatatttggagatgactacgatacacgtgacggttcatgtattcgagattacatttatgtcttggaccttgcataggctcatgttaaaggaatgaactacctatttcaagaagacagttcttctcaaatctttaacttaggaactaaaaaaggctatactatcaaagaaatctttaaaactgctgaagaattactaaattaaaaaatcccacacgagattgttgctcgacgtgctagtgacccagccagtgtcctagcagatgcttcaaaagctaaacaatatcttgattggacggctagctactccctcgaagatattattttatcagattaaggtaaaaacattttagaataggataacaaaggagaaagtcttgttgctctctccttgttgtatttattaaattgtcagaaattttattgacagattagaaaaataatgttacatttatatccgcagatatctttcgataccaaatctacatgaaaggacggggtatgaaactttctcattatttaattggcttacttctactcctagtctttctctctattagcattgggaccagtgatttttcatggggaaagctatttgatttcgaccagcagacctggctcctctttcaagagtcccgtctcccaagaactatcagtattctcctgactgcctctagtatgagtatggcaggccttctcatgcagactattacccaaaatcagtttgctgcaccgagtacagttggaacgactgaagccgccaaactgggaatggtgctgagcctttttgtctttccatcggctagtctgacccacaagatgctcttcgcttttgtttcatccatcgtattcaccctcttcttcctagcctttatgaccatttttactgtaaaggaaaggtggatgttgcctctgattgggatcatctatagcgggattatcggctcagtcacagaagttatcgcctatcgtttcaatctggttcagagtatgactgcctggacccagggctccttctccatgattcagacccatcagtatgagtggctcttcttaggcctcatcatcctgataaccgtttggaaattatcccaaaccttcaccatcatgaatctagggaaagaaaccagcgagagtttggggatttcctactccctacttgaaaaactggccctctttctggtggcgctaacgacaagcgtcaccatgattaccgtgggtggcctaccatttctcggagttatcgttcccaatcttgttcgcaagcgctatggagataatctaagtcaaaccaaactcatggtcgcactggttggtgccaatctagttctggcttgcgatatcctatcccgagttctgattaggccctatgagttgtctgtcagtctcttgctaggaatcatcggtagtctcgtctttatcctacttctctggagagggggacgaaaagatgcagactaaaagcaaacataccaagctcttctggattctcattattcttgccatcggagcttgtcttctctacttttggcccatcactcacttgtcagcctttgcttggaagttgcgttcccaaaagatcatcgtttatctcttggtagccatcgcgactgggatttcgaccattagttttcaaaccctgacggaaaatcgcttcctgacgcctagtattttgggaattgaatccttctacgtcctactacaaaccctactactggtttttgaaagcaagtttcttcaacttggcaaatcccctatcttagaattcctagtcttacttcttgtccagtccctcttctttctcgccttacaaggttacttgaagacactgatgaagcaagacctggtcttcgtcctgctgatctgtctagcgctcagaagtctctttcgaaatatcagcaccttccttcaagtcctaatggatccaaacgaatacgataaactgcaaaatagtctttttgcctcctttcaacatctcaacacttccatcctagccatcggttctctgatcatcctcgctttgacaatctttttctttcgaaaagcagtcgttctagatgtcttgcacctgcaaagagaaacggctcagatattgggactcgatgttgaaaaagaacagaaagagctcctctggggaatcgtgcttttgacctcaacggccactgccttggtaggacctatggccttcttcggctttatactggccaacctcacctacctgattgtcaaagactatcagcacaagttactctttatagtggccattctggttggatttattagcttaaccttggggcaagccttgattgaacgagtctttgcactggaaattcgtatcagtatgatcattgagagtgtgggtggcttcttattctttatcttactatataggaggtctcgtcagtgaaactggaaaacattgacaaatccattcaaaaacaggatattttgcaaggcatttcgcttgaagtcagtcctcaaaaactgacagcctttattggtccaaatggtgctggaaaatcgactctcctctccatcatgagcaggctaaccaagaaagatcagggagttctcagtatcaaaggacgtgaaatcgagagctggaattcgcaagaactggcccaagaactaaccatcctaaaacagaaaatcaattaccaagccaaattgactgttgaagaactggtcagttttggacgttttccctacagccgaggtcgacttagatcagaagactgggaaaaaatccgagaaactctgaactatttagaactgaccaacttaaaagaccgctacatcaatagcctgtcaggggggcaactccagcgcgtctttatcgctatggtactggcccaggatacggactttatcttgctggacgaaccactcaacaatctcgatatcaagcaaagcgtcagcatgatgcagattcttcgacgactggtggaggaactcggcaagaccattatcatcgtcctccacgatatcaacatggccagtcagtatgcagatgaaattgtcgccttcaaggacggccaggtctttagcaagggaagaaccgatcaaatcatgcaggctgacctgctcagtcaactttatgagattcccatcacgctagctgatatcaatgacaaaaagatctgtatctatagctagtaacataaaagctcaagttagagaaccttcagtctcttagtcaataagattaagagactccctaaatcgttatcacattttaaaaaggagaaattatgaaaacatcccttaaactttatttcactgccctagtggccagcttcttgctcctacttggtgcatgtagtacaaactcaagcactagtcagacagagaccagtagctctgctccaacagaggtaaccattaaaagttcactggacgaggtcaaactttccaaagttcctgaaaagattgtgacctttgacctcggcgctgcggatactattcgcgctttaggatttgaaaaaaatatcgtcggaatgcctacaaaaactgttccgacttatctaaaagacctagtgggaactgtcaaaaatgttggttctatgaaagaacctgatttagaagctatcgccgcccttgagcctgatttgattatcgcttcgccacgtacacaaaaattcgtagacaaattcaaagaaatcgccccaaccgttctcttccaagcaagcaaggacgactactggacttctaccaaggctaatatcgaatccttagcaagtgccttcggcgaaactggtacacagaaagccaaggaagaattgaccaagctagacaagagcatccaagaagtcgctactaaaaatgaaagctctgacaaaaaagcccttgcgatcctccttaatgaaggaaaaatggcagcctttggtgccaaatctcgtttctctttcttgtaccaaaccttgaaattcaaaccaactgatacaaaatttgaagactcacgccacggacaagaagtcagctttgaaagtgtcaaagaaatcaaccctgacatcctctttgtcatcaaccgtacccttgccatcggtggggacaactctagcaacgacggtgtcctagaaaatgcccttatcgctgaaacacctgctgctaaaaatggtaagattatccaactaacaccagacctctggtatctaagcggaggcggacttgaatcaacaaaactcatgattgaagacatacaaaaagctttgaaataagccacttcaaactcaatttaatactcaatgaaaatcaaagagcaaactaggaaactagccgcaggttgctcaaaacactgttttgaggttgcagatgaaagctgacgcggtttgaagagattttcgaagagtataaaattttgacattctaaaacgcatcctatcaagttcactcaaacctgataggatgcgtttttatcattttgaaatctatttaccccacctcattccccttcttgatttcgtttaagggaaaagcggtctggaacggggtccttacctgttttcgaccagggatgacaacgtaaaatccgagccaagcccatcaatacccccttaaacccatgtttttcaatagcctgaatcatgtagttggaacaagtcagctcaaagcgacaagagggtggaaaga

SEQ ID NO: 2 - DNA, 975 bp, Salmonella pneumoniae
  CDS (1)..(975)
atgaaaggacggggtatgaaactttctcattatttaattggcttacttctactcctagtctttctctctattagcattgggaccagtgatttttcatggggaaagctatttgatttcgaccagcagacctggctcctctttcaagagtcccgtctcccaagaactatcagtattctcctgactgcctctagtatgagtatggcaggccttctcatgcagactattacccaaaatcagtttgctgcaccgagtacagttggaacgactgaagccgccaaactgggaatggtgctgagcctttttgtctttccatcggctagtctgacccacaagatgctcttcgcttttgtttcatccatcgtattcaccctcttcttcctagcctttatgaccatttttactgtaaaggaaaggtggatgttgcctctgattgggatcatctatagcgggattatcggctcagtcacagaagttatcgcctatcgtttcaatctggttcagagtatgactgcctggacccagggctccttctccatgattcagacccatcagtatgagtggctcttcttaggcctcatcatcctgataaccgtttggaaattatcccaaaccttcaccatcatgaatctagggaaagaaaccagcgagagtttggggatttcctactccctacttgaaaaactggccctctttctggtggcgctaacgacaagcgtcaccatgattaccgtgggtggcctaccatttctcggagttatcgttcccaatcttgttcgcaagcgctatggagataatctaagtcaaaccaaactcatggtcgcactggttggtgccaatctagttctggcttgcgatatcctatcccgagttctgattaggccctatgagttgtctgtcagtctcttgctaggaatcatcggtagtctcgtctttatcctacttctctggagagggggacgaaaagatgcagactaa

SEQ ID NO: 3 - protein, 324 aa, Salmonella pneumoniae
MKGRGMKLSHYLIGLLLLLVFLSISIGTSDFSWGKLFDFDQQTWLLFQESRLPRTISILLTASSMSMAGLLMQTITQNQFAAPSTVGTTEAAKLGMVLSLFVFPSASLTHKMLFAFVSSIVFTLFFLAFMTIFTVKERWMLPLIGIIYSGIIGSVTEVIAYRFNLVQSMTAWTQGSFSMIQTHQYEWLFLGLIILITVWKLSQTFTIMNLGKETSESLGISYSLLEKLALFLVALTTSVTMITVGGLPFLGVIVPNLVRKRYGDNLSQTKLMVALVGANLVLACDILSRVLIRPYELSVSLLLGIIGSLVFILLLWRGGRKDAD

SEQ ID NO: 4 - DNA, 954 bp, Salmonella pneumoniae
  CDS (1)..(954)
atgcagactaaaagcaaacataccaagctcttctggattctcattattcttgccatcggagcttgtcttctctacttttggcccatcactcacttgtcagcctttgcttggaagttgcgttcccaaaagatcatcgtttatctcttggtagccatcgcgactgggatttcgaccattagttttcaaaccctgacggaaaatcgcttcctgacgcctagtattttgggaattgaatccttctacgtcctactacaaaccctactactggtttttgaaagcaagtttcttcaacttggcaaatcccctatcttagaattcctagtcttacttcttgtccagtccctcttctttctcgccttacaaggttacttgaagacactgatgaagcaagacctggtcttcgtcctgctgatctgtctagcgctcagaagtctctttcgaaatatcagcaccttccttcaagtcctaatggatccaaacgaatacgataaactgcaaaatagtctttttgcctcctttcaacatctcaacacttccatcctagccatcggttctctgatcatcctcgctttgacaatctttttctttcgaaaagcagtcgttctagatgtcttgcacctgcaaagagaaacggctcagatattgggactcgatgttgaaaaagaacagaaagagctcctctggggaatcgtgcttttgacctcaacggccactgccttggtaggacctatggccttcttcggctttatactggccaacctcacctacctgattgtcaaagactatcagcacaagttactctttatagtggccattctggttggatttattagcttaaccttggggcaagccttgattgaacgagtctttgcactggaaattcgtatcagtatgatcattgagagtgtgggtggcttcttattctttatcttactatataggaggtctcgtcag

SEQ ID NO: 5 - protein, 318 aa, Salmonella pneumoniae
MQTKSKHTKLFWILIILAIGACLLYFWPITHLSAFAWKLRSQKIIVYLLVAIATGISTISFQTLTENRFLTPSILGIESFYVLLQTLLLVFESKFLQLGKSPILEFLVLLLVQSLFFLALQGYLKTLMKQDLVFVLLICLALRSLFRNISTFLQVLMDPNEYDKLQNSLFASFQHLNTSILAIGSLIILALTIFFFRKAVVLDVLHLQRETAQILGLDVEKEQKELLWGIVLLTSTATALVGPMAFFGFILANLTYLIVKDYQHKLLFIVAILVGFISLTLGQALIERVFALEIRISMIIESVGGFLFFILLYRRSRQ

SEQ ID NO: 6 - DNA, 618 bp, Salmonella pneumoniae
  CDS (1)..(618)
atgagcaggctaaccaagaaagatcagggagttctcagtatcaaaggacgtgaaatcgagagctggaattcgcaagaactggcccaagaactaaccatcctaaaacagaaaatcaattaccaagccaaattgactgttgaagaactggtcagttttggacgttttccctacagccgaggtcgacttagatcagaagactgggaaaaaatccgagaaactctgaactatttagaactgaccaacttaaaagaccgctacatcaatagcctgtcaggggggcaactccagcgcgtctttatcgctatggtactggcccaggatacggactttatcttgctggacgaaccactcaacaatctcgatatcaagcaaagcgtcagcatgatgcagattcttcgacgactggtggaggaactcggcaagaccattatcatcgtcctccacgatatcaacatggccagtcagtatgcagatgaaattgtcgccttcaaggacggccaggtctttagcaagggaagaaccgatcaaatcatgcaggctgacctgctcagtcaactttatgagattcccatcacgctagctgatatcaatgacaaaaagatctgtatctatagctag

SEQ ID NO: 7 - protein, 205 aa, Salmonella pneumoniae
MSRLTKKDQGVLSIKGREIESWNSQELAQELTILKQKINYQAKLTVEELVSFGRFPYSRGRLRSEDWEKIRETLNYLELTNLKDRYINSLSGGQLQRVFIAMVLAQDTDFILLDEPLNNLDIKQSVSMMQILRRLVEELGKTIIIVLHDINMASQYADEIVAFKDGQVFSKGRTDQIMQADLLSQLYEIPITLADINDKKICIYS

SEQ ID NO: 8 - DNA, 966 bp, Salmonella pneumoniae
  CDS (1)..(966)
atgaaaacatcccttaaactttatttcactgccctagtggccagcttcttgctcctacttggtgcatgtagtacaaactcaagcactagtcagacagagaccagtagctctgctccaacagaggtaaccattaaaagttcactggacgaggtcaaactttccaaagttcctgaaaagattgtgacctttgacctcggcgctgcggatactattcgcgctttaggatttgaaaaaaatatcgtcggaatgcctacaaaaactgttccgacttatctaaaagacctagtgggaactgtcaaaaatgttggttctatgaaagaacctgatttagaagctatcgccgcccttgagcctgatttgattatcgcttcgccacgtacacaaaaattcgtagacaaattcaaagaaatcgccccaaccgttctcttccaagcaagcaaggacgactactggacttctaccaaggctaatatcgaatccttagcaagtgccttcggcgaaactggtacacagaaagccaaggaagaattgaccaagctagacaagagcatccaagaagtcgctactaaaaatgaaagctctgacaaaaaagcccttgcgatcctccttaatgaaggaaaaatggcagcctttggtgccaaatctcgtttctctttcttgtaccaaaccttgaaattcaaaccaactgatacaaaatttgaagactcacgccacggacaagaagtcagctttgaaagtgtcaaagaaatcaaccctgacatcctctttgtcatcaaccgtacccttgccatcggtggggacaactctagcaacgacggtgtcctagaaaatgcccttatcgctgaaacacctgctgctaaaaatggtaagattatccaactaacaccagacctctggtatctaagcggaggcggacttgaatcaacaaaactcatgattgaagacatacaaaaagctttgaaataa

SEQ ID NO: 9 - protein, 321 aa, Salmonella pneumoniae
MKTSLKLYFTALVASFLLLLGACSTNSSTSQTETSSSAPTEVTIKSSLDEVKLSKVPEKIVTFDLGAADTIRALGFEKNIVGMPTKTVPTYLKDLVGTVKNVGSMKEPDLEAIAALEPDLIIASPRTQKFVDKFKEIAPTVLFQASKDDYWTSTKANIESLASAFGETGTQKAKEELTKLDKSIQEVATKNESSDKKALAILLNEGKMAAFGAKSRFSFLYQTLKFKPTDTKFEDSRHGQEVSFESVKEINPDILFVINRTLAIGGDNSSNDGVLENALIAETPAAKNGKIIQLTPDLWYLSGGGLESTKLMIEDIQKALK

SEQ ID NO: 10 - DNA, 27540 bp, Salmonella pneumoniae
tgtcgataagcacataagtgttgaaattgagctggatgagatggatttgacaagtgcggagagcaaagcaacatatgctcaaatcaaagaatatgtttggaataaatttgaattaaaagtttcgacattatatattgcacagataaaaaagaaatgtggaatagaattacgagaacattacaacaagtctaaaaaggataaacaaattattccacagtgtacacctgaaaaagaagaagccatcatggatgctttgagacacttcaaaatgatttaatagaaaagaatgacagtatatgactttctgcatttattacattcctacttggtataggaacagctattattcctttcttgcaaggtatcaattagaaaataggctcaatataaagattgataggatcatttttatatttaaaggagcgttgaaatgattgataaaggcaacaaaaaattttaggataaatttgctaagttgtatgcctcttttatgaaaaaagataaagaggtttatgataaagtttgtgaatatcttagtcctcatttgaataaagatatggaggtgcttgaacttgcttgttggtttcgtgtcataacagttatagaggcaaatagttatgtaaatataaggagttcaagacttctaccaaagtttaaaactcaaaaaataaatagttggtgtgctgcttacaatatccattttaataatggatattgtaagcagcacacctaatgaatttaaagattctttaaagagtcttattttgtgatgaaaatttaatatgtaaatctcagacgatagaaattaaaaactctatcgtcttttttatactcaaaattaggaggtaaaaatggtaaggataagaggtcccacttaaaacaatttatggcaaaataaggacggaataacacaacaaattctctaaaacaaatcactaaatcaatgtaagattgaatgaaatcaatatttatgctataattaaataaatttaatgaagaaaaaaagagggatattatggcacttaactataaaccattatggatacagttagcaaaaaaaggactaaagaaaacagatgtaatagctatggcaggacttacaacaaatgttatggcacaaatgggaaaggataaaccaattacatttaagaatttagaaagaatatgtaaggctttatcttgcactcctaatgatattattagttttgaagataattttagtgacgaggaatagaaaatgactttaaggacagaagatcaagttagggattatgcaagagaagtataggctttaatgaagttgaagaaaacatcaatcaaggtactggtcaaataactacttttaatcaattaggcttcaagggatattcaaataagccagatggttggtatttacctaaaaatatgaatgatgtagcaataatccttgaaacaaaatcagaagaaagagatattagcaaacaaatttttattgatgagttaatgaaaaatatagacataatttaactaaaaataaaaactagatccttttttgaaaaaattatattattaaatttgtaactgtatctattgacaatgataattattatcgatacaatagacttgaaatatgtttaaggagtttttatgaaaaacaaattttttctaatagctattttagctatgtgtatagtttttagcgcttgttcttctaattctgttaaaaatgaagaaaatacttctaaagagcatgcgcctgataaaatagttttagatcatgctttcggtcaaactatattagataaaaaacctgaaagagttgcaactattgcttggggaaatcatgatgtagcattagctttaggaatagttcctgttggattttcaaaagcaaattacggtgtaagtgctgataaaggagttttaccatggacagaagaaaaaatcaaagaactaaatggtaaagctaacctatttgacgatttggatggacttaactttgaagcaatatcaaattctaaaccagatgttatcttagcaggttattctggtataactaaagaagattatgacactctatcaaaaattgctcctgtagcagcatacaaatctaaaccttggcaaactttatggagagatatgattaaaattgattcaaaagccttaggtatggaaaaagaaggtgatgagttaatcaaaaatactgaagctcgtatatccaaagaattagaaaaacatccagaaatcaaaggaaaaatcaaaggaaaaaaagtattatttactatgattaatgctgcagatacatcaaaattctggatttatactagcaaagatccaagagcaaattatttaacagatttaggtctagttttccctgaatcattaaaagaatttgagagtgaagatagttttgcaaaggaaatttctgcagaagaagcaaataagataaatgatgctgatgtaatcataacttatggtgatgataaaactcttgaagctttacaaaaagatcctcttttaggtaaaataaatgcaattaaaaatggtgccgttgctgtaattccagataatacaccgttagcagcctcatgcactccaacaccactttcaataaactatactattgaagaatacctaaatcttttaggaaatgcatgcaaaaatgcgaaataaaaaacaaataaacctaggcataatttttataatctgcctaggtcttcttattacaatatttttgtcattaaagcttggaacaaaagaaattaatatcagagattttttagcagcttttggaatgggtaatacaaatgatgattttattaaatcaattatatataaaagaatacctagaactatttttgcaattttagcaggttctagtcttgccataagcggtgtattgatgcaatcagttactagaaacccaatagctgatccaggtatactcggtataaacacaggagcaagtcttagtgtagtaattggtctttcttttttaggaatttcatcaagcataagccatataagttttgcaatcattggtggcttagtaagtgcaatttttgtatacgcgattgctgtaagcggaaaagcaggccttacccctataaaacttgccttatcaggaacttgtgttagtatggctttaagcagttttgtaagttttttaattttaccgaataataacgtcttagacaaatttagattttggcaaataggtagccttggagcagctacattatcttctatatctacactactaccttttataattttaggtcacttgatagctatatttatttcatcagatttaaacgctttagctatgggtgatgaaatggctgttggtcttggagttaatgttaataggataagatcacttgcaataattgcaagtgtgcttttatgttcaagtattactgcaattggtggacctattggcttcgtaggtcttatagttcctcacttttgtggcttatttataagcaaagatatacgcacaatgaccatttcttcatcttttataggtgcagagctcttgcttatatgtgatataatcggccgtatgttaggtaaaccaggtgaaattgaagtagggataattactgcaataatcgggggtccagtacttatttatgtaactatgaaaaatagaggggttaataactaatgcaaaatttaattataggtattcaaaaaagaaaaaatagaataacactattttcctcactatttttattaataataatcagtctatcatttttcattttacttatcggagatgaaagttattctttttcaactttgattaaagtcttaaatagtgaaactgttcctggagctagtttttcgattatggaaattagattaccaaaattattagcaggaattatagctggctggtcttttggattggcaggatttatctttcaaactatgttaagaaatcctcttgcaagtcctgatataatcggtgtcacaagttcttcatctattgcagcggtcttttgcatattggtattaaaaacaaatagtttaactactggaattatttcaataacttgtggactaacatcatctttaatattatttttactagctaaaaaagatggtttttcagcagcaagactgataatattaggtattggttttcaagctgtcacaagagcaggcacctcatttttattgttgaaagtagcaagatatgaattacaagaagttatgagatggctcagtggctctttatcttttacaaagttagatgacatacctcttgttctaatagtaagtattattgctactatattagttttattttttaataaaagactagaaattattgaacttggtgaagaaatagcaatcggacttggagcaaatcccgagctttcaaggcttgttttaattttttgcgctgtatctttaactgctttttctacttcaattacaggaccaatagcttgtatatcttttttagctggtcccatagccttaaatattggcaagaaaagaagcccaatattagctggattggttggaattttactagttttgttatcagacatattctctcaaaatattttaccagctagatatccagtaggtgttgtaactggcttgttaggttcaccatacttaatatacttactaataaaaatgaacaggaggaatatataatgaaaggtctgtggtcaaataatcttacctgcggttatgatgaaaaaataatcttggaaaatataaatataaaaatacctgaagaaaaaatatcagttattattgggtcaaatggttgtgggaaatcaacactcattaaaaccttgtctcgacttataaagccattagagggagaagtattgcttgataataaatcaattaattcttataaagaaaaagatttagcaaaacacatagctatattacctcaatctccaataatccctgaatcaataacagtagctgatcttgtaagccgtggtcgtttcccctacagaaagccttttaagagtcttggaaaagatgaccttgaaataataaacagatcaatggttaaggccaatgttgaagatctagcaaataacctagttgaagaactttctgggggtcaaaggcaaagagtatggatagctctagccctagcccaagatacaagtatcctacttttagatgagccaactacttacttggatatctcatatcaaatagaactattagacctcttgactgatctaaaccaaaaatataagacaaccatttgcatgattttgcacgatataaatctaacagcaagatacgctgattacctatttgcaattaaagaaggtaaacttgttgcagagggaaagcctgaagatatactaaatgataaactagttaaagatatctttaatcttgaagcaaaaattatacgtgaccctatttccaattcgcctctaatgattcctattggcaagcaccatgttaactcttaataattgaatattggaattcatttaaaagtcgcaaattttgaactgaccctcaaaagttaggccaaaaaactaactaaaggaggtcagttttttaatggcaaatacagcacagaatttaagataaaattagtaaaagaatatcatgaatgaaaaataggatatagagaataacagcaatattaaaaagatcaggactgataataaacaaaaagaaagtacaacgactagttcaaaagctaaaacttcaagtaaaaagttattcaagaaaatctagaaaatactcatcctacaaaggacaggtaggaaaaatatcagacaacaaaataaaaagaaatatttattaccgaaaaaaaggggaaatcgagttaagtaccaacaggctcatctattccaaaaagatatttactcgatggaaaaactcctagaattagtgctacaaatattgacaatggtatcttaggttactacgaagatatagatgataaaaattatagagtttttgaaaattttatatctgtttcatttttaggagcagtcttttatcataaatataaagcaagcctagatatgaaaattcattgtttaaaattaaaaaataaagaattgaataaagaagtagcattctatttaacttcaattattaggcaagctttaaaaaatactgaatataaagatcaaatatctagcacagtattgccagatatcaaaattaaattaccgatagacagtagaggtactccagattggaattacatggaaagatatagagatagataaagagagagagggagagatagataaaaagtccacccccattttttatggagaattcaaaaatgtaaggttaagcaaagaagaatataaaaatctaaaagaaaaattaaactcacacacagatataatgataaataaactatccagatacatggaaagcagtggtaagacctatcaaaaccactatgtgacaatcttaaaatggtatgaagaagataaagacaaactaagacagaaaggtttaaataaaaaaatgaattatgatgtaggagaatctttatagataaaaagaggtggaaaggcactattaaagactttaagtctaaaaaggtatcaaagtatgccagagataaaaaaatataaatatgacactttaaaggcgattagaaaattaaagaaatgctagaagacttttagatgacagttaccaagagggtctaattttcactgctatttatgcagtgggaattggacctttttatataagcaaattctatttcaaaattggttaccaagaaagacttcttttcactgttaagtataagagggtaatttatagaattttgttacatcaagataaatattttctgatggtcaaattttaaaaagattaataactctcgataaattttgaaccttaacaattgaatagaccaagacaagacgttaattatttttggagcgtaataacctatccgtcaagatctttcggctgaataattcggcaaaaaaagttctgctaagcaaaaagaaagtgagcgataagtaagagtttgaatatagggagggataccctatccgctatgaagaaaatgagggtaatgatacttctaaggttgaagccggctcatccggaatagaggcggaagtgagattcctatgttgctaatccaaagcacttgtcaatgtcaaaaaacttaattaaacaaataattattagatataaaactttagaggaggtatatattggacaatgactggaatggattggctgatttaatagcaaatctgattgcaaaatatgctggtgctttagatttagataatctacccgatccaacgccagcaaaaaatcaagagatgaaaaatagttttgacatggcaaaaacacaaattgagacggactaataaaagtgttataatatacttgaaatgatagtccaaacttaaattaaaaaaattatatatgatatagaaaaattatattgaggttaatactatgtcaaaagaaaaaataaaagtatacctctatacacgagtatctacgtcaatacagatagaggggtattcgttagaggcacaaaaatcacgaatgaaagcttttgctatttacaacgattatgaaattgttggagagtatgaagatgccgggaagtctgacaagtctattgaaggtagaatacagttcaatcgaatgatggaagatataaaatctggaaaagatggagtatcttttgttcttgtgtttaagttatcaagatttgcaagaaatgccgctgatgttctttctacacttcatataatgcaggattatggagttaatcttatctgcgttgaagatggcattgattcatctaaagatgctgggaaactaatgatttctgttttatcagctgtggctgaaatagaaagagaaaacatcagtattcaaacaatggaaggtcgcattcaaaaagcaggggaaggaaaatggaatggtggctttgctccgtatggatataaacttgaagacggcaagctgtttataaatgaggaagaggcagttgcaataagaacgattttcgaccagtatgtaaatactacgataggagccaatgggatatctaaatacttagagaatcatggaattagaaaaatcccaagacagaatggtaagaatcctttgtttgatgcaggtcttataagaaagatattaaagaatcctgtatataatgggaaaatagcatttggaagaagaactttagaaaaagttcatggtacaagaaatgaatataagcaggttgaacaagatgaatatctaatatctgaagggatacatgaagctatagtttccgatgaagtttggcaagctgctcaggttaagctaaaatctcaagcaaagaaatatgagcatgtgaataaaggaaaagatacacgcacacacttgctttctggaattgtaaaatgcccgatatgtggagtgggaatgtttgggaacaagtgtatcaagaaaaagaaagatggcacaaagtataaagatttttattactatggttgtaaccataggcagatgataagaggtaataagtgtacattcagtaagcaaattagagaagaattgttagatgatgctgttgcagaggtgattgtcaagatagtaagtaatccgaaatttgcttctatgatgcaagaaaaaatcaacatgaaggtggatacctctgaaatagaaaaagaaatagataattaccaaaaggaattgagaaagagtcattctacaaagtttaagctaattgaggaaatagataatttagatgttgaagataagcattataagagaaggaaacaggatttagatgatagactatatcgtatgtatgacaaactagatgaattagaatcatcactaattgatgcgaaagcaaagaaacagactattgaagctgaaaagcttacaggagataacatatataaggttctgatctattttgataagctctataaagtcatgaatgatgtagagcgtaggcagttaatttcagctttgatttctgaaattcaagtttatgaagaaaaacaatctaacggacaatggctaaaatccattacttttaagctaccaattatagaagaaaatctcaacataggtttggacaatgatgaacaagttgaatgtgtaagtttattagagaaacgtagttaatcctcaaaaggttccccgaaccttttgagtcccactgacgcatcacgctgaggcggtatcactgcttgtacgagttgaagcatcagcaaagtaaaagtagatgttcagccaatgcgacgaggtgagaccgaaggtcgataaggtgtgttctgggtataagactcgtaaagtaagtaggaaatatgatgagatgatacagagagcctgaattggtcaggtgacgtgcttatttacattagcctgataatgaaaatggagagattatcgatggttatattggaattgtaccaaaatgattattctaaagatttagtagcatttgattccatagaagatgggaaagcctttgtagctcaaatcccgggatacactctggaaacagaagatggttttgaagtggagtattttaatcctaaaaacataccggattatatggaaattatttttaacggaaatattgttcctttatctaaatttatgttcgatcctgaggaaaatgtaaacattatttggaaagagatttcaaatttatccctcaaaaatgatagagtgatcgaaggatattcaaaaattgatgcatatgtcgttaacaaccatgaagttaaagcctatgtcgaaacaagagaaacgaactatcgtaaagcaaaagacttcttagaaagccgcggatatgaaatcgacagaagctttttcggaagtgaagacggtgaagcgattctttacagaaaaaaagggatcgaagtttggcatttcttgtgtcacttagacccaatgtttgtagagattgaagatgtagaaggatatgttaaagaagaagttggagagattcaatagtcaaccaaagttccttgtgagtatatacctttgctagtaaaagagtcaaaacgttataacgaagggggggaagaaaacccttgagtgtaagtctttttcttgtttagagggaaagtattagagtagccgctcataatttatactagatttgataactgatttgagacagattttatttctataaatattgtgaaaattttttaataccaaagatttaaagttactgtgttaaaaatcctctccacttttaaactaatgtattataaaagccagttgacactagtgttacacctgtgatagaattaacttgaggtgatgaaaatggcaaccgataaaaatagaataatgattagcttggatgataaaaatctagagaaacttgaaaatttagtagaagatgctagagatagaaggggaatgcgactaacaaagtcccaggttattgaattactcctaaataccgttgattatttcgatgatattatgggggctatttactcaaaaaaataagagcaaaattgctattattttttacaaattggtgtaacactagtgatataggagtgatgaaatggttgtttatattagacaaagtaaattaccaagtgaggtttcaattaataaatacaatgctcaagtaggtgcctacttacaaggagaagaagttattttatatcaatctttttctgaaataaaagagttaacaagtgaagatatagttgtagattatataatggagactagagcattactaaaaatgatgggcttaaacgttccggttcatgactatcctattgagcttaaagagttttatggtcgaaagatttatgctggtattttaggagagattgtgaatatacctgataattggggaaaatttattaagcctaaagctggttcaaaagtctttactggaagagttgttaatggaacccatgatttaataggtattggtctacctttcgactatcctatatggattagtgaggttgtagaattcatagctgaatggcgttgttttgtgttagatggtcgcgtattagatgttcgaccctatacaggtgattatcatgcacaatttgatgcaagtgtaattgatgaagcgatatcatgttggaaagatgcgccaatagcttatggactagatatcggtgttactcgcgatggcagaacacttgttgttgaagtaaatgatggttatgcattgggaaattatggcctatctcctttaaaatcaatcaattttcatagagctaggtggaaagaaatggtaaaaccctattttgaaaaaaatgaaatttttaaaatccaacaggatgttattttctaaaataaatatggtagataaccaatataaaaagcatcaaaatgaacaaggaggaatctgatgcttggagcaattattggagatattgtcggttctgtttacgaatggaacaatatcaaaacaaaggactttcctttatttcggaaggactgcttttttacagatgacacggttatgacctgtgctgttgcagaagcaattatgaatggtggacaaaaagatgacttcattgacgcgatgaagaaatatggcagaatgtatccgaatgctgattacggtgctcggtttaatgcatggctaaacagcgataaccgtgagccttataatagctttgggaatggatcagctatgcgtatttctccatgtgcttggatcatggactgtggtttttatgcgaaaactggtatatggccatcatctagaggacttacgagtctttctgcagaggtaactcataatcatccagaaggtgtcaaaggcgctatggctacagctgatgctatctttctatgtcgtttttactttggtggttattgtagagagtacgagcaatcaattaacgacaatcctacagagtgtaaaagacgaattaaggattatatagaaaaagaatacgattacaatctatctcaaactttagatgaaatccgtcctaactatcgttttaacgaaacatgtcaggaaactgtccctcaagccattatcgcctttcttgaaagtagagacttcgaagatgcgataagaaatgccatctcacttggtggcgacagtgatactctcgcagcaatcacttgtagcatagcagaggcagcttacggtattcctgattggatcaaggataaggcctattcttacttggatgaacccttaaaggatgtagttaggcgatgggaaaatagaataaaagcgtattaatctccagtacgtcagatttctcttttctgtattttcattgtataataggattacatttacttgcatacgacgaaatgtaaacatttctgtttaaaattaaggataaaatcaatgaaaaaagcaatggtaattatcaaccctacttctggtggcgagaaggctttggattacaaagaaaagctggagaataaagcaaaagaatactttgaatatgttgaaaccaaaattaccgaaaaagcgctggatgcaacacattttgctgaagaagcttctcgtgagcagtatgatgcagtggttgtgtttggtggagatggaactgtcaatgaagtcatttcaggtattgatgagagagactacattcctaagttagggattatcccaggcggtacgggtaacctcattacaaaacttttggaaatcaatcaagacatcgatggcgcaattgacgaactggattttgatttaaccaataagattgatatcggtaaagcaaatgacaactattttggttatatctttagtatcggttctctgcctgaggcgattcacaatgttgaaatagaggacaaaacaaaattcggtattttaacctatgctgtaaataccatgaagtctgtcatgacagatcaggtctttaacattaaggttgagacagaaaatggaaattatgttggtgaagctagccatgttttggtccttttgacaaattacttcgctgataagaaaatctttgaagaaaacaaggacggctatgccaacattttgattctgaaagatgcctctatattctccaaattatccgtcattcctgatttattaaaaggggatgttgtcgcaaatgataatatcgagtatatcaaagcgcgtaatattaaaatctcttcagatagtgaattggagtcagatgttgacggagataaatcagataacctacctgtagaaatcaaagtcctagctcagcgagtagaagtattttcaaaaccgaaagaggattagtatatagagaaagccttttttaaggctttttgtatactttaaaagatagttcctttaacaacggacattccttgcaaatagttttacaaaaatagtatactggattcattgagtttgaaaacgtttgcgtaaaatttgaatgaatactttaggagacaaattgatggaattgagtgctatttaccataggcctgagtcggagtatgactatctttataaggataagaaactccatattcgaattcgaactaagaaaggggacattgaaagcatcaacttgcactatggggacccttttatctttatggaggagttttatcaggatacaaaagaaatggtcaagataacttctggtaccttatttgaccattggcaggttgaagtgtcagttgactttgcacgtatccagtatctctttgagctcagagatacagaaggtcaaaatattttgtatggcgataaagggtgtgtggaaaattctctagaaaatcttcatgcaattgggaatggatttaagttgccttagcttcatgagattgatgcctgcaaggttcctgactgggtttcaaatacggtatggtatcagatatttcctgaaagatttgccaatggcaatgctctattaaacccagaagggactttagactgggattcatctgtcacacctaagagcgatgatttctttggtggtgatttacaggggattattgatcatatgaattacttgcaagacttgggtattactggactatatctttgtcccatctttgaatctacaagcaatcacaagtacaatacgacagattactttgaaattgaccgtcattttggagacaaggagacctttcgggaactggtggatcaagcgcatcatcgtggcatgaaagtcatgctggatgcggtatttaatcatattggttcgcaatctcttcaatggaaaaatgtcgtcaaaaatggtgaacagtctgcttataaggattggttccatattcaacaattcccagtgacaactgaaaagctagttaataagagagacttaccctatcatgtttttggtttcgaggactatatgcctaagctaaatacagccaatccagaggtcaagaattatcttttaaaggttgcgacttattggattgaagagtttaatatcgatgcttggcgtttggatgtggctaatgagattgaccatcagttctggaaggattttcgtaaggcagttttagctaaaaatcctgatctttatatcctaggagaagtctggcatacatctcagccttggctaaatggagatgagttccatgccgtcatgaattatcctttatctgatagtatcaaggactatttcttacgaggaattaagaagacagaccagttcatcgatgaaatcaatggagagtctatgtattacaagcagcagatttcagaggtcatgtttaatctcttggattcacatgatacagagcgaatcctgtggacggccaatgaagatgttcaactggttaaatcagccttagcctttctctttttacaaaaaggaacaccgtgcatttattacggaaccgagctagccttgactggaggaccagatccagattgtcgtcgttgtatgccttgggaacgtgtatcaagtgacaatgatatgctgaactttatgaagaggctgattaaaattcggaaatacgcgtcagtaatcatttcgcatggcaagtatagccttcaagaaatcaactctgatctagtagctctggaatggaaatacgaaggacggatcctcaaagcaatattcaaccaatcaacagaagattatcttttagagaaagaagcagtagcactagcaagcaattgccaagaattggataatcagcttgtcatctctccagatggatttatgattttctaaaaactagttgatgaagattatggtacatttcataccttatatagtataataaggctagttactaaacttgtaaaggagaacttaaatgaattgtagaggacatgaaacaagacaaagaattgttagagattttgaagttcagcctaaagcacatattaagctgttagcaaatcaacaaaaacatagtgatgcaggagcaactattgaagatgaatattatgtatttatcgctgagagtaaaattgatggcaagaaggaagttattcagtgttgcatgggtgcggcaagggattttttagaactaattaatcacaaagggctacctctttttaatccgcttgtaggtgattctcatgtaaataatagacaagaatatgacaatacagggagtggaaatttataacctgaaaagtggaatgaaactgcaaagcagctttataatgctataatgtggttgattattttatggaatgctaagccggatacacctttatttaattttaaagacgaagtaattaagtataaaacatatgagccttttgaaagcagtataaaaagagtaaatactactataaagaatggtagtaaagggaaaactctgactgagatgattaatggctacagagcggataacgatattagagatgaaatttgtaactttaatattctgaaaaataaaattcgtgatatgaaaaaccaacaaggaaatacaatggaatcttacttttagttattgttgaattttgggtattctataaaatatcctaattgagatttaaatagtagactatacaatatagttaaaatatcagtaaaaacaacactttattgaggtattggatacgctttgctaatagcctaataatcacatgtggagtgttgctacaacgaaaaaggtgataatccttgatttcaagctattttataagcattttgtctttgtagataaaggcaattttgacaataaaaatcctaaaaggtgaatcgttatagatgtatttgtagatatcgtttgcgcatcgaaaaaattaatacaagaataaatatttatagctctttaggtgacttttatagaagtaaagtttaggatagaaaaacaagaaataacgcaccatttttggtgcgttatgcttttttatgctataatggatttataaaaataaaggagtttgctatgattggaaagaacataaaatccttgcgtaaaacacatgacttaacacaactcgaatttgcacggattgtaggtatttcacgaaatagtctgagtcgttatgaaaatggaacgagttcagtctctaccgaattaatagacatcatttgtcagaagtttaatgtatcttatgtcgatattgtaggagaagataaaatgctcaatcctgttgaagattatgaattgactttaaaaattgaaattgtgaaagaaagaggtgctaatctattatctcgactctatcgttatcaagatagtcagggaattagcattgatgatgagtctaatccttggattttaatgagtgatgatctatctgatttgattcatacgaatatctatctagtagaaacttttgatgaaatagagagatatagtggctatttggatggaattgaacgtatgttagagatatctgaaaaacggatggtggcctaatggaaatccaagattatactgatagtgaattcaaacatgctttagcaaggaatcttcgttcactgacaagaggaaaaaagtccagtaagcaacctatagcgattttgcttggagggcaaagtggtgccggtaagactacaattcatcgtattaaacagaaagaatttcaaggaaatattgttatcatagatggtgatagttttcgttctcagcatccacactatttagaactgcagcaagaatatggcaaagacagtgtagaatataccaaagattttgcaggaaaaatggtagagtctttagtaacaaaattgagtagtttgagatacaatcttttgatagagggaactttacgaacagttgatgttccaaagaaaacagcacaactcttgaaaaataagggatatgaagtacaattggccttaattgcgacaaagcctgaattgtcgtatctaagtactcttatccgttatgaagaactgtacattatcaatccaaatcaagcacgcgcaactccaaaagaacatcatgatttcattgtaaatcatctagttgataacacacgaaaattggaagaactagctatctttgaaagaattcaaatttaccaacgagatagaagttgtgtatatgattcaaaagaaaatacaacttcagcagcagatgttcttcaagagttactctttggggagtggagtcaggtagagaaggagatgttgcaggtgggggaaaagagacttaatgaattacttgaaaaataaacaattgatatttttaggagaatagaaatgagagggtttaataacaagataaagtctgtttatcaagaactaacaaattccaaagagaaattcggtagctttcacaagactttaattcatttgcatacacctgtttcttatgattacaagctattttctaattggactgcaacgaaatatagaaaaattactgaagatgaactatatgatatattttttgaaaataagaaaataaaagttgataagacaattttttttagtaattttgataaggttgttttttctagttcaaaagaatatattagttttcttatgttagcagaggcaatcataaaaaatggaatagaaatagttgtagtaactgatcataatactaccaaaggtattaaaaagttacaaatggcagtctcaatcataatgaaaaattatccgatttatgatatacatcctcatattttacatggagtagaaattagtgcagcagataaattgcatattgtatgtatatatgattatgaacaagaatcatgggttaatcaatggttaagtgaaaatattataagtgagaaagatggaagttatcaacattcactgactataatgaaggatttcaataatcaaaaaatagttaactatattgctcatttcaatagttatgacattttgaaaaaaggttctcacttatcaggtgcatataaacgaaaaattttttctaaagaaaatacacgattttggagtttaatattaactcgaaagaatcttcgcaacaacttgatattctctataaagaagttggtgtattaagtttgggacaaaaagttgtagccatgcttgattttttattagcatatagtgattattctaaagacttcagaccattgattattgatcagcctgaagacaatctagacaatcgttatatttacaggcatttagttcagcagtttagagatgtgaaagctcaacgtcaaattattttagcaacacataatgctacaattgtaacaaattctatgacagatcaagttgttattatggagtcagatggagttaacggatggattgaatcacagggatatgttagtgaaaaatatataaaaaatcatatcatcaatcaattagagggaggaaaagattccttcaagcataaaatgtctatatatgagacggctttatcagagtagagtcagaaaaagtaggttagaaatttagcctacttttttctttgtccgacaggcatagtgtacatctgaggtccaagtcctctgtggatatttgctgcagatgaaaccaatagcgactcctaagcctgaatatcgtgaggtaggggggataggaaggaattagcgaaatcaaggttctacaaacagaatcgtgacttgaagccatatatagcggatgaggaactctaaaatccaaataggtgtcgtaacctatatacgtaaattacgagagtaaactaggaaagatgtacggcttattccgtgagcgtttaggacgtagtacaacgaatcatgggagtcagctgaacacatagtattgaagaaatttctgtaatggaaatggagcgaagaagtgaacaattaaatgaatacctctctaattaaatttgtcaattctaattcctggtatgaaaagacagtgacctgaaaatgtaaacgatgggagctgatcataaatataggacggtacatgcagtggtgttagagattagtccttacttgatttgtgataacttccccaaatttcttctgctatacttttctcaacttttaaaaatccaactaagaattttacctgggggtttgggggcggagcactaagttatcttatcgttagctgtcaaaactggtaggttttgataggctggcgatatgatttttgggatattgtggacacaatatctgagctcgcaaagccttacaagaatgaaaatcagttgttggaaaagtgtactgacattgtatggtagctcacattgtcagtacaagtattttggaaaggaagtagcagtatgaaacgagatgtgcgtgatattcggaaacaatttcgtttaacagaagcagaagaaaagcaaattctagctttgatgagagagcggggagagactaatttctctgattttcttcgtaaaagtttactttcctctgatttacaaaaacagatggagacatggtttgccctctggcaatcccaaaaactagaacaaatcagtcgtgacgttcatgaagttttaatcttggcacagtcagaacgtcaagtcacccaagagcatgtatctattctcttaacgtgcgtgcaggaattgattcaagaggttgcaaacaccatacccctcagtaaagaatttcgtgagaagtacatgaggtaagcacatggaacatcgttaccgaaccaatctcaagaaagtgtttttgtctgatagtgagttgaaccaactaaatataaatatcgatcaaagtggttgtaaatccttttctgaatatgcgagacgaactctactcgatcctggtatgaattttatcacgattgacacaaacggttaccaagatttagtgtttgagttaaagaggattggcaataatatcaaccagattgctcgaagtgttaatcaatctcagttaatttctggtgaagaattgcaggagttgaaaaaaggaattggtgaattgataaaagaagttgataaggaatttaatctgcaagcgcagaagctaaaggagttccatggtcatcactaaacactttgccattcacggaaagagttaccgcagaaagcttatcaagtacattctcaatcctgagaaaaccaataatcttgccttggtgtcggactatggcatgaagaattttctggactttcctagctatgaggaaatggtgcagatgtatcatgaaaatttcatcagcaacgatacgctttacgattttcgccacgacaggatggaagaaaatcaacgaaaaatacacgctcaccacatcattcagtctttctcgccagaggatcatatcactcctgaacaaatcaatcggataggttatgagactgtgaaggaattaactggtggcaaatttcgttttatcgttgcgacccatgttgataaagaccacctgcacaatcacatcattatcaattcagtagatagcaattctgacaaaaagctcaagtgggactacaaggtggagcgaaatcttcgcatgatttctgaccgtttttctaaaatcgcaggtgctaaaatcattgagaaccgctattctcaccagcggtatgaagtctatcgtaagactaatcacaagtatgaactcaagcagcgactctattttttgatggaacattctagggactttgaggatttcaaaaagaatgctccgctactacatgtggagatggatttccgtcacaagcatgccaccttttttattacggactcaactatgaaacaggtggtgcgtggcaagcaactcaatcgcaagcagccttacacagaagaattttttaagaactactttgccaaaagagaaatagaaagtctcatggaatttttattgctgaaagttgagaatatggatgatttacttcagaaagcaaaactttttggactaactatcaatcctaaacaaaagcatgtttcttttcaatttgcaggagtggaggtaaaggagacagagctagaccagaaaaatctttatgatgtagagtttttccaagattattttaaaaatagaaaagattggcaagctccagaaactgaggatttcgttcaactttatcaagaagaaaagttatccaaagaaaaagaacttccaagcgatgagaagttctgggagtcctatcaagagttcaagagtaacagagatgccgttcatgaatttgaggtggagttgtcactcaatcaaattgaaaaagtagtggatgatggaatttacgtcaaggtcaagtttggtattcgtcaggagggacttatctttgtgccgaacatgcagcttgatatggaagaggataaggtgaaggttttcatcagggaaaccagctcctactatgtctaccacaaagacgctgccgagaaaaattgttatatgaaaggtcgaaccttaattagacagttcagctatgaaaatcaaaccattccattacgcagaaaagcgacagtcgatatgattaaagagaagattgcggaagtggatgctttgattgaactggaagtagaaaatcaatcttatgtcacgattaaagatgagttagtgcatgaactagcagcgtctgaattgagaatcaatgagttgcaagaacgaatgtcaaccttgaatcaagtagcagaatatctactggcttcagttgaaagtaagcaagaaatgaaattaaatctttcaaaactgaatataactgagaatatcagtgctaatattgttgagaaaaaattgaagagcctggggaatcaactggaattggaaaggggcaggtatgaaaagatggtagtcaggctagataaatttataaatcgcttaaatacaggactttcaaaaggagatgggatagattttcaaaaataggaatctagccaataattatatacaatgtgtatcttttgtaaaatatatttgaaatttagatttaatcatgttacaattaacattgaaatcaaatagaaaaggaagatgcaaacgtgggtacactattagcaacaagattaaaaaatagacgaaaagaattaaaaatgtctcagcgagaattagctgagggtatctgtaaacaggggcaaattagtcgattagagagtggagagttcacccctggagcggattttttacatgcccttgctaagaaattaaaagttagtatggactacttttttgatgagcaggttgtagaggaagtcaatgagttatcagagtttaagaagttagcacagacatttatcacaaatcgtaattatgaatctttaaaatatatatatgaattggaaaatgtaaaagcccatcgcttatctctagcagataaattttacatggaatggataaagtctcttatagatttttatttttatggacagaaagaagaggcaattacacgattagagaatatgctacctcagttaaatgtatctgatttgaactaccttcaagtttcaaacactctattcaatttttattatgatattgaagatttagaacgttttaatgagattagagacaagctagagcatcaagtgaatcagcttaatttaaacacaattgaagagttagaactatcaattaagttcaattataacgtttgtcgatacctatggttacagaaaaatattgaagaagctattacgaaaatcacagctacgataaagcaatgtaaggagtacaggacaacttatctattagctgatttgtatttattgatgggaagtgtaagtgaaaatttttcttctaaaagttcagtaaaagaatattttgagactgctcattttctctacaaacttgaggaaaacatgtcaatggctttgaaagtcgaacattattttgcagatataacagaataaggaatacggtgttctagtgagcatcgttttttaaatctaatttgaatacaaaaaagtattaaataaaaaaaaataaaaaaagattataaaaatacttgacaagctaattttgaagtgataaaataaatgcaagaaaacgaaaggagaggtatatgagaagtttgtttaggaaaattgttgctttgttagtcataggattaatcctattagggactgctggaggaactcaagttcataagatggctagagggattgatccaggtcctgcgaatggtatttatagataggagattatagtcacttaaaaatcaaaaaattataaaatttttgttttacttaaatagatattttatgatgggcgaagtttttataactagttcctataagtacagaaacctattttaatttactaatttcaaaacaattttttgagatatatgtaattaaatctaaattaagctttaaaaaattatttttagcaatttgattttgaaagtgtcatgcagcaatatgaaagcgtagaatagaggtgtttccaatgtacaagtaagttttgattccaaataaagtaatagaaaaggagctatataatgagtagtcaaatgaaagctttcttgaatgaacttcaaggaaatatggaagttgttggagaattttttgataatcctgaaaaggtaattagaaaatttggaattgtcggaagagagaaggaagcattgctaatccgtgacttaaatgatatggatgattatgctctttcaattcaaaatagcgttgcatcgccttctggggcacatagtagcacttgccattttgttagacaagatttacaatccgcataattgttaatatataaaacataaaatttcaattttcaattagggataatagaattccttactatgatatagaaagtgaattaattaggaaatcttagtcagaggtttctgattaattcattttttatatattaagagtgatgtgaaatgttcaaattgtgagactactctcggattgttgtatagaaatttaaaattttattgtgagctttttaaggaagagtggccgatatgaaaaataaatacgatgttaaaagaattattccggatgaattatcagaatcactagatatttttctaaaaaattattctgaaactggattgtcagattataatacttatctcttttatggttttatcttgaagtcctataaattgcctcgagaaaatagatatagcataaaattattggtgaaggaattacaaaatagaggactgaaagttacattgattatcaacatttattatcacgctttaaattgtttggctttaaatgatggtctaaaaatttatggagaagatttcttaatatgaattacgaaaagtatttaaactatttggactatgaaactgaaatagaagacgcataccataatttattattagaatataaaattagtgataattttagtgatgaacattggttatataatttgccgtctaatatcacccaatctaagggatttaaaatacacctgagcgcttcaatattaaatgctaatttggttgctaaaaaattctttgattttatattttctagagagaagaagataaatttcaaaattttggttagcattaaagaactttctttgcaaaatacagggcttaatggttattcacaagtcgggaaattcattacaatttatccaaaagataataaagagtttcaacgtttgttacacaaattagaaatattgtataaaggtgtaaagggtgtaaacataccatcagatttcagatttcaacttagcgaggttgtatattacagatatggggagttcgtaaaagatagcactttcaaggataaaagagataagaaaatcccttcaaatgtgaatgttccaataagggattactatattccaagatataacactattcctgatcaatatattattttggaagtgatttctaaaaatgccaaaggtggagtttataaagtatttaatactcaaaagcgagtttattctttattaaaagaagctagtgatttatcgttggttgattttacaaatagagattctgtgaatcgtttaataaatgaaagagaaatattagtagaattggaaaaggaggaatttactccaaaagtttttaacgatttttacataaagaattcgtattttgtggaatttgagtttattattgcagataaattaagcgagtataatatggttagtggctcgtatgattggtttttaacattaattgattatatggagataataaactccaaatataatctttcctatagagatttatcatttaataatatacttattgataatagaaataatatttatattatagactttgagcatgccctaaggaaagaaactcaaattgaagaaaaaaagttgcctctatttggaactccagggttttatgaaaccaacttaaatttattgaataatcaaccagaggatattatgggattggttttgttattatattggagtcagaatttagatgattttaacgtcttttcaaaaatgagttttttagaagcgatggagtatgcaatgaattttaattcgcgtcgcttagacacacttgataaatcagattggctttatgatatttataaaaaagcgttcaattataaatatgatactttttctgaattaaaaaaagattttatagaggtattaaaataaaggtaggggtgttatgaattgtttcttgaaaatgaataatgtaagtgttcgttatgatgacgtaatagctttaaaagatataactttacaaataaataagggagatttcattggcttattaggttcaaatggtgcaggtaaatctacgttaattaattctattgtaggttttcaagagatttatttaggagaaatagagtattgtgataaagatttgatagttagttctcaaccttttgctcatttaggctttactcctcaaaccacagtaattgatttttatactactgtgaaggacaatgtaatattggggctgaaccttgctggaaagtttgggaaaaatgctgagaagttgtgtcaaatagccttagaaattgttgggttagctgataaaaaaaataatttggtagaaacattgtcaggtggacaactgcaacgcgtccagattgctagagcaatagctcataatccagatttttatattttagatgaacctaccgttggtttagatactgaatctgccgaaaaatttttaatgtatttaaaagataagagtttggaaggaaaaactattatcatatcttcacatgacataaatctactcgaaaagttttgtaaaaaaatactttttttacaaaatggctccatatcattttttggtgatatgcgtgactttgtagataattcaactatcaaattaaatttttcaatgcagaatagaatttctagatatcaaattgaatttttagaaaattttagatttaaagttcacatcgaagataatgatagttttacaatagaagtccctatagaagaaaagatcttagatgttatcaatgaggtaggaaaagcatgtgaaattaaaaacttttcaacaagtaaattaaccttacaagaaagttatttgcaaagaataggaggagaaaaatgaaggctgatcaattaaggcacaaatcggacttaggtttaagaggtctagcgattattgctaaaaatgagattattgctttttttagaagtaaaggtttaattatttctcagtttctacaaccaatcttatatgttgtttttataataataggattaaattcttcgataaagaacattcagtttaatgatataaaaacctcttatgcagaatatacaatcattggtgttatagctttattgataatcgggcagatgactcaagttatttatagggtgacaatagataaaaaatatgggctacttgctcttaagttatgcagtggagttcgtcctttatattatattttagggatgagtatctattctatattagggttgatagttcaagaaattattatatatataattacgttagcgtttgagataaatatcgcaatggatagatttttttatacagttttgttatctattgttgttttattattttgggactcccttgcaattttacttacaatgtttatcaatgattacagaagacgtgatattgtaatacgttttgtactaacaccgcttggttttacagctcctgttttctacttaatagattctgctcctagtattgtgagatggattggtcagttaaatcccttaacttatcaattaactattttgagaaacttttattttaaaaattcaacaactttggaattagttttcttattgttaacatcattacttgtccttatatctgtatcttttattataccaaagataaaattgatactgatagaaagataaaagttgggtcatccaacttttttgttgtctcccgaaaaccactagctatgctagt

SEQ ID NO: 11 - DNA, 1026 bp, Salmonella pneumoniae
  CDS (1)..(1026)
atgaaaaacaaattttttctaatagctattttagctatgtgtatagtttttagcgcttgttcttctaattctgttaaaaatgaagaaaatacttctaaagagcatgcgcctgataaaatagttttagatcatgctttcggtcaaactatattagataaaaaacctgaaagagttgcaactattgcttggggaaatcatgatgtagcattagctttaggaatagttcctgttggattttcaaaagcaaattacggtgtaagtgctgataaaggagttttaccatggacagaagaaaaaatcaaagaactaaatggtaaagctaacctatttgacgatttggatggacttaactttgaagcaatatcaaattctaaaccagatgttatcttagcaggttattctggtataactaaagaagattatgacactctatcaaaaattgctcctgtagcagcatacaaatctaaaccttggcaaactttatggagagatatgattaaaattgattcaaaagccttaggtatggaaaaagaaggtgatgagttaatcaaaaatactgaagctcgtatatccaaagaattagaaaaacatccagaaatcaaaggaaaaatcaaaggaaaaaaagtattatttactatgattaatgctgcagatacatcaaaattctggatttatactagcaaagatccaagagcaaattatttaacagatttaggtctagttttccctgaatcattaaaagaatttgagagtgaagatagttttgcaaaggaaatttctgcagaagaagcaaataagataaatgatgctgatgtaatcataacttatggtgatgataaaactcttgaagctttacaaaaagatcctcttttaggtaaaataaatgcaattaaaaatggtgccgttgctgtaattccagataatacaccgttagcagcctcatgcactccaacaccactttcaataaactatactattgaagaatacctaaatcttttaggaaatgcatgcaaaaatgcgaaataa

SEQ ID NO: 12 - protein, 341 aa, Salmonella pneumoniae
MKNKFFLIAILAMCIVFSACSSNSVKNEENTSKEHAPDKIVLDHAFGQTILDKKPERVATIAWGNHDVALALGIVPVGFSKANYGVSADKGVLPWTEEKIKELNGKANLFDDLDGLNFEAISNSKPDVILAGYSGITKEDYDTLSKIAPVAAYKSKPWQTLWRDMIKIDSKALGMEKEGDELIKNTEARISKELEKHPEIKGKIKGKKVLFTMINAADTSKFWIYTSKDPRANYLTDLGLVFPESLKEFESEDSFAKEISAEEANKINDADVIITYGDDKTLEALQKDPLLGKINAIKNGAVAVIPDNTPLAASCTPTPLSINYTIEEYLNLLGNACKNAK

SEQ ID NO: 13 - DNA, 1020 bp, Salmonella pneumoniae
  CDS (1)..(1020)
atgcatgcaaaaatgcgaaataaaaaacaaataaacctaggcataatttttataatctgcctaggtcttcttattacaatatttttgtcattaaagcttggaacaaaagaaattaatatcagagattttttagcagcttttggaatgggtaatacaaatgatgattttattaaatcaattatatataaaagaatacctagaactatttttgcaattttagcaggttctagtcttgccataagcggtgtattgatgcaatcagttactagaaacccaatagctgatccaggtatactcggtataaacacaggagcaagtcttagtgtagtaattggtctttcttttttaggaatttcatcaagcataagccatataagttttgcaatcattggtggcttagtaagtgcaatttttgtatacgcgattgctgtaagcggaaaagcaggccttacccctataaaacttgccttatcaggaacttgtgttagtatggctttaagcagttttgtaagttttttaattttaccgaataataacgtcttagacaaatttagattttggcaaataggtagccttggagcagctacattatcttctatatctacactactaccttttataattttaggtcacttgatagctatatttatttcatcagatttaaacgctttagctatgggtgatgaaatggctgttggtcttggagttaatgttaataggataagatcacttgcaataattgcaagtgtgcttttatgttcaagtattactgcaattggtggacctattggcttcgtaggtcttatagttcctcacttttgtggcttatttataagcaaagatatacgcacaatgaccatttcttcatcttttataggtgcagagctcttgcttatatgtgatataatcggccgtatgttaggtaaaccaggtgaaattgaagtagggataattactgcaataatcgggggtccagtacttatttatgtaactatgaaaaatagaggggttaataactaa

SEQ ID NO: 14 - protein, 339 aa, Salmonella pneumoniae
MHAKMRNKKQINLGIIFIICLGLLITIFLSLKLGTKEINIRDFLAAFGMGNTNDDFIKSIIYKRIPRTIFAILAGSSLAISGVLMQSVTRNPIADPGILGINTGASLSVVIGLSFLGISSSISHISFAIIGGLVSAIFVYAIAVSGKAGLTPIKLALSGTCVSMALSSFVSFLILPNNNVLDKFRFWQIGSLGAATLSSISTLLPFIILGHLIAIFISSDLNALAMGDEMAVGLGVNVNRIRSLAIIASVLLCSSITAIGGPIGFVGLIVPHFCGLFISKDIRTMTISSSFIGAELLLICDIIGRMLGKPGEIEVGIITAIIGGPVLIYVTMKNRGVNN

SEQ ID NO: 15 - DNA, 1008 bp, Salmonella pneumoniae
  CDS (1)..(1008)
atgcaaaatttaattataggtattcaaaaaagaaaaaatagaataacactattttcctcactatttttattaataataatcagtctatcatttttcattttacttatcggagatgaaagttattctttttcaactttgattaaagtcttaaatagtgaaactgttcctggagctagtttttcgattatggaaattagattaccaaaattattagcaggaattatagctggctggtcttttggattggcaggatttatctttcaaactatgttaagaaatcctcttgcaagtcctgatataatcggtgtcacaagttcttcatctattgcagcggtcttttgcatattggtattaaaaacaaatagtttaactactggaattatttcaataacttgtggactaacatcatctttaatattatttttactagctaaaaaagatggtttttcagcagcaagactgataatattaggtattggttttcaagctgtcacaagagcaggcacctcatttttattgttgaaagtagcaagatatgaattacaagaagttatgagatggctcagtggctctttatcttttacaaagttagatgacatacctcttgttctaatagtaagtattattgctactatattagttttattttttaataaaagactagaaattattgaacttggtgaagaaatagcaatcggacttggagcaaatcccgagctttcaaggcttgttttaattttttgcgctgtatctttaactgctttttctacttcaattacaggaccaatagcttgtatatcttttttagctggtcccatagccttaaatattggcaagaaaagaagcccaatattagctggattggttggaattttactagttttgttatcagacatattctctcaaaatattttaccagctagatatccagtaggtgttgtaactggcttgttaggttcaccatacttaatatacttactaataaaaatgaacaggaggaatatataa

SEQ ID NO: 16 - protein, 335 aa, Salmonella pneumoniae
MQNLIIGIQKRKNRITLFSSLFLLIIISLSFFILLIGDESYSFSTLIKVLNSETVPGASFSIMEIRLPKLLAGIIAGWSFGLAGFIFQTMLRNPLASPDIIGVTSSSSIAAVFCILVLKTNSLTTGIISITCGLTSSLILFLLAKKDGFSAARLIILGIGFQAVTRAGTSFLLLKVARYELQEVMRWLSGSLSFTKLDDIPLVLIVSIIATILVLFFNKRLEIIELGEEIAIGLGANPELSRLVLIFCAVSLTAFSTSITGPIACISFLAGPIALNIGKKRSPILAGLVGILLVLLSDIFSQNILPARYPVGVVTGLLGSPYLIYLLIKMNRRNI

SEQ ID NO: 17 - DNA, 795 bp, Salmonella pneumoniae
  CDS (1)..(795)
atgaaaggtctgtggtcaaataatcttacctgcggttatgatgaaaaaataatcttggaaaatataaatataaaaatacctgaagaaaaaatatcagttattattgggtcaaatggttgtgggaaatcaacactcattaaaaccttgtctcgacttataaagccattagagggagaagtattgcttgataataaatcaattaattcttataaagaaaaagatttagcaaaacacatagctatattacctcaatctccaataatccctgaatcaataacagtagctgatcttgtaagccgtggtcgtttcccctacagaaagccttttaagagtcttggaaaagatgaccttgaaataataaacagatcaatggttaaggccaatgttgaagatctagcaaataacctagttgaagaactttctgggggtcaaaggcaaagagtatggatagctctagccctagcccaagatacaagtatcctacttttagatgagccaactacttacttggatatctcatatcaaatagaactattagacctcttgactgatctaaaccaaaaatataagacaaccatttgcatgattttgcacgatataaatctaacagcaagatacgctgattacctatttgcaattaaagaaggtaaacttgttgcagagggaaagcctgaagatatactaaatgataaactagttaaagatatctttaatcttgaagcaaaaattatacgtgaccctatttccaattcgcctctaatgattcctattggcaagcaccatgttaactcttaa

SEQ ID NO: 18 - protein, 264 aa, Salmonella pneumoniae
MKGLWSNNLTCGYDEKIILENINIKIPEEKISVIIGSNGCGKSTLIKTLSRLIKPLEGEVLLDNKSINSYKEKDLAKHIAILPQSPIIPESITVADLVSRGRFPYRKPFKSLGKDDLEIINRSMVKANVEDLANNLVEELSGGQRQRVWIALALAQDTSILLLDEPTTYLDISYQIELLDLLTDLNQKYKTTICMILHDINLTARYADYLFAIKEGKLVAEGKPEDILNDKLVKDIFNLEAKIIRDPISNSPLMIPIGKHHVNS

SEQ ID NO: 19 - DNA, 1680 bp, Salmonella pneumoniae
  CDS (1)..(1680)
atgtcaaaagaaaaaataaaagtatacctctatacacgagtatctacgtcaatacagatagaggggtattcgttagaggcacaaaaatcacgaatgaaagcttttgctatttacaacgattatgaaattgttggagagtatgaagatgccgggaagtctgacaagtctattgaaggtagaatacagttcaatcgaatgatggaagatataaaatctggaaaagatggagtatcttttgttcttgtgtttaagttatcaagatttgcaagaaatgccgctgatgttctttctacacttcatataatgcaggattatggagttaatcttatctgcgttgaagatggcattgattcatctaaagatgctgggaaactaatgatttctgttttatcagctgtggctgaaatagaaagagaaaacatcagtattcaaacaatggaaggtcgcattcaaaaagcaggggaaggaaaatggaatggtggctttgctccgtatggatataaacttgaagacggcaagctgtttataaatgaggaagaggcagttgcaataagaacgattttcgaccagtatgtaaatactacgataggagccaatgggatatctaaatacttagagaatcatggaattagaaaaatcccaagacagaatggtaagaatcctttgtttgatgcaggtcttataagaaagatattaaagaatcctgtatataatgggaaaatagcatttggaagaagaactttagaaaaagttcatggtacaagaaatgaatataagcaggttgaacaagatgaatatctaatatctgaagggatacatgaagctatagtttccgatgaagtttggcaagctgctcaggttaagctaaaatctcaagcaaagaaatatgagcatgtgaataaaggaaaagatacacgcacacacttgctttctggaattgtaaaatgcccgatatgtggagtgggaatgtttgggaacaagtgtatcaagaaaaagaaagatggcacaaagtataaagatttttattactatggttgtaaccataggcagatgataagaggtaataagtgtacattcagtaagcaaattagagaagaattgttagatgatgctgttgcagaggtgattgtcaagatagtaagtaatccgaaatttgcttctatgatgcaagaaaaaatcaacatgaaggtggatacctctgaaatagaaaaagaaatagataattaccaaaaggaattgagaaagagtcattctacaaagtttaagctaattgaggaaatagataatttagatgttgaagataagcattataagagaaggaaacaggatttagatgatagactatatcgtatgtatgacaaactagatgaattagaatcatcactaattgatgcgaaagcaaagaaacagactattgaagctgaaaagcttacaggagataacatatataaggttctgatctattttgataagctctataaagtcatgaatgatgtagagcgtaggcagttaatttcagctttgatttctgaaattcaagtttatgaagaaaaacaatctaacggacaatggctaaaatccattacttttaagctaccaattatagaagaaaatctcaacataggtttggacaatgatgaacaagttgaatgtgtaagtttattagagaaacgtagttaa

SEQ ID NO: 20 - protein, 559 aa, Salmonella pneumoniae
MSKEKIKVYLYTRVSTSIQIEGYSLEAQKSRMKAFAIYNDYEIVGEYEDAGKSDKSIEGRIQFNRMMEDIKSGKDGVSFVLVFKLSRFARNAADVLSTLHIMQDYGVNLICVEDGIDSSKDAGKLMISVLSAVAEIERENISIQTMEGRIQKAGEGKWNGGFAPYGYKLEDGKLFINEEEAVAIRTIFDQYVNTTIGANGISKYLENHGIRKIPRQNGKNPLFDAGLIRKILKNPVYNGKIAFGRRTLEKVHGTRNEYKQVEQDEYLISEGIHEAIVSDEVWQAAQVKLKSQAKKYEHVNKGKDTRTHLLSGIVKCPICGVGMFGNKCIKKKKDGTKYKDFYYYGCNHRQMIRGNKCTFSKQIREELLDDAVAEVIVKIVSNPKFASMMQEKINMKVDTSEIEKEIDNYQKELRKSHSTKFKLIEEIDNLDVEDKHYKRRKQDLDDRLYRMYDKLDELESSLIDAKAKKQTIEAEKLTGDNIYKVLIYFDKLYKVMNDVERRQLISALISEIQVYEEKQSNGQWLKSITFKLPIIEENLNIGLDNDEQVECVSLLEKRS

SEQ ID NO: 21 - DNA, 588 bp, Salmonella pneumoniae
  CDS (1)..(588)
atgaaaatggagagattatcgatggttatattggaattgtaccaaaatgattattctaaagatttagtagcatttgattccatagaagatgggaaagcctttgtagctcaaatcccgggatacactctggaaacagaagatggttttgaagtggagtattttaatcctaaaaacataccggattatatggaaattatttttaacggaaatattgttcctttatctaaatttatgttcgatcctgaggaaaatgtaaacattatttggaaagagatttcaaatttatccctcaaaaatgatagagtgatcgaaggatattcaaaaattgatgcatatgtcgttaacaaccatgaagttaaagcctatgtcgaaacaagagaaacgaactatcgtaaagcaaaagacttcttagaaagccgcggatatgaaatcgacagaagctttttcggaagtgaagacggtgaagcgattctttacagaaaaaaagggatcgaagtttggcatttcttgtgtcacttagacccaatgtttgtagagattgaagatgtagaaggatatgttaaagaagaagttggagagattcaatag

SEQ ID NO: 22 - protein, 195 aa, Salmonella pneumoniae
MKMERLSMVILELYQNDYSKDLVAFDSIEDGKAFVAQIPGYTLETEDGFEVEYFNPKNIPDYMEIIFNGNIVPLSKFMFDPEENVNIIWKEISNLSLKNDRVIEGYSKIDAYVVNNHEVKAYVETRETNYRKAKDFLESRGYEIDRSFFGSEDGEAILYRKKGIEVWHFLCHLDPMFVEIEDVEGYVKEEVGEIQ

SEQ ID NO: 23 - DNA, 759 bp, Salmonella pneumoniae
  CDS (1)..(759)
atggttgtttatattagacaaagtaaattaccaagtgaggtttcaattaataaatacaatgctcaagtaggtgcctacttacaaggagaagaagttattttatatcaatctttttctgaaataaaagagttaacaagtgaagatatagttgtagattatataatggagactagagcattactaaaaatgatgggcttaaacgttccggttcatgactatcctattgagcttaaagagttttatggtcgaaagatttatgctggtattttaggagagattgtgaatatacctgataattggggaaaatttattaagcctaaagctggttcaaaagtctttactggaagagttgttaatggaacccatgatttaataggtattggtctacctttcgactatcctatatggattagtgaggttgtagaattcatagctgaatggcgttgttttgtgttagatggtcgcgtattagatgttcgaccctatacaggtgattatcatgcacaatttgatgcaagtgtaattgatgaagcgatatcatgttggaaagatgcgccaatagcttatggactagatatcggtgttactcgcgatggcagaacacttgttgttgaagtaaatgatggttatgcattgggaaattatggcctatctcctttaaaatcaatcaattttcatagagctaggtggaaagaaatggtaaaaccctattttgaaaaaaatgaaatttttaaaatccaacaggatgttattttctaa

SEQ ID NO: 24 - protein, 252 aa, Salmonella pneumoniae
MVVYIRQSKLPSEVSINKYNAQVGAYLQGEEVILYQSFSEIKELTSEDIVVDYIMETRALLKMMGLNVPVHDYPIELKEFYGRKIYAGILGEIVNIPDNWGKFIKPKAGSKVFTGRVVNGTHDLIGIGLPFDYPIWISEVVEFIAEWRCFVLDGRVLDVRPYTGDYHAQFDASVIDEAISCWKDAPIAYGLDIGVTRDGRTLVVEVNDGYALGNYGLSPLKSINFHRARWKEMVKPYFEKNEIFKIQQDVIF

SEQ ID NO: 25 - DNA, 855 bp, Salmonella pneumoniae
  CDS (1)..(855)
atgcttggagcaattattggagatattgtcggttctgtttacgaatggaacaatatcaaaacaaaggactttcctttatttcggaaggactgcttttttacagatgacacggttatgacctgtgctgttgcagaagcaattatgaatggtggacaaaaagatgacttcattgacgcgatgaagaaatatggcagaatgtatccgaatgctgattacggtgctcggtttaatgcatggctaaacagcgataaccgtgagccttataatagctttgggaatggatcagctatgcgtatttctccatgtgcttggatcatggactgtggtttttatgcgaaaactggtatatggccatcatctagaggacttacgagtctttctgcagaggtaactcataatcatccagaaggtgtcaaaggcgctatggctacagctgatgctatctttctatgtcgtttttactttggtggttattgtagagagtacgagcaatcaattaacgacaatcctacagagtgtaaaagacgaattaaggattatatagaaaaagaatacgattacaatctatctcaaactttagatgaaatccgtcctaactatcgttttaacgaaacatgtcaggaaactgtccctcaagccattatcgcctttcttgaaagtagagacttcgaagatgcgataagaaatgccatctcacttggtggcgacagtgatactctcgcagcaatcacttgtagcatagcagaggcagcttacggtattcctgattggatcaaggataaggcctattcttacttggatgaacccttaaaggatgtagttaggcgatgggaaaatagaataaaagcgtattaa

SEQ ID NO: 26 - protein, 284 aa, Salmonella pneumoniae
MLGAIIGDIVGSVYEWNNIKTKDFPLFRKDCFFTDDTVMTCAVAEAIMNGGQKDDFIDAMKKYGRMYPNADYGARFNAWLNSDNREPYNSFGNGSAMRISPCAWIMDCGFYAKTGIWPSSRGLTSLSAEVTHNHPEGVKGAMATADAIFLCRFYFGGYCREYEQSINDNPTECKRRIKDYIEKEYDYNLSQTLDEIRPNYRFNETCQETVPQAIIAFLESRDFEDAIRNAISLGGDSDTLAAITCSIAEAAYGIPDWIKDKAYSYLDEPLKDVVRRWENRIKAY

SEQ ID NO: 27 - DNA, 936 bp, Salmonella pneumoniae
  CDS (1)..(936)
ttgcatacgacgaaatgtaaacatttctgtttaaaattaaggataaaatcaatgaaaaaagcaatggtaattatcaaccctacttctggtggcgagaaggctttggattacaaagaaaagctggagaataaagcaaaagaatactttgaatatgttgaaaccaaaattaccgaaaaagcgctggatgcaacacattttgctgaagaagcttctcgtgagcagtatgatgcagtggttgtgtttggtggagatggaactgtcaatgaagtcatttcaggtattgatgagagagactacattcctaagttagggattatcccaggcggtacgggtaacctcattacaaaacttttggaaatcaatcaagacatcgatggcgcaattgacgaactggattttgatttaaccaataagattgatatcggtaaagcaaatgacaactattttggttatatctttagtatcggttctctgcctgaggcgattcacaatgttgaaatagaggacaaaacaaaattcggtattttaacctatgctgtaaataccatgaagtctgtcatgacagatcaggtctttaacattaaggttgagacagaaaatggaaattatgttggtgaagctagccatgttttggtccttttgacaaattacttcgctgataagaaaatctttgaagaaaacaaggacggctatgccaacattttgattctgaaagatgcctctatattctccaaattatccgtcattcctgatttattaaaaggggatgttgtcgcaaatgataatatcgagtatatcaaagcgcgtaatattaaaatctcttcagatagtgaattggagtcagatgttgacggagataaatcagataacctacctgtagaaatcaaagtcctagctcagcgagtagaagtattttcaaaaccgaaagaggattag

SEQ ID NO: 28 - protein, 311 aa, Salmonella pneumoniae
LHTTKCKHFCLKLRIKSMKKAMVIINPTSGGEKALDYKEKLENKAKEYFEYVETKITEKALDATHFAEEASREQYDAVVVFGGDGTVNEVISGIDERDYIPKLGIIPGGTGNLITKLLEINQDIDGAIDELDFDLTNKIDIGKANDNYFGYIFSIGSLPEAIHNVEIEDKTKFGILTYAVNTMKSVMTDQVFNIKVETENGNYVGEASHVLVLLTNYFADKKIFEENKDGYANILILKDASIFSKLSVIPDLLKGDVVANDNIEYIKARNIKISSDSELESDVDGDKSDNLPVEIKVLAQRVEVFSKPKED

SEQ ID NO: 29 - DNA, 1188 bp, Salmonella pneumoniae
  CDS (1)..(1188)
atgaattacttgcaagacttgggtattactggactatatctttgtcccatctttgaatctacaagcaatcacaagtacaatacgacagattactttgaaattgaccgtcattttggagacaaggagacctttcgggaactggtggatcaagcgcatcatcgtggcatgaaagtcatgctggatgcggtatttaatcatattggttcgcaatctcttcaatggaaaaatgtcgtcaaaaatggtgaacagtctgcttataaggattggttccatattcaacaattcccagtgacaactgaaaagctagttaataagagagacttaccctatcatgtttttggtttcgaggactatatgcctaagctaaatacagccaatccagaggtcaagaattatcttttaaaggttgcgacttattggattgaagagtttaatatcgatgcttggcgtttggatgtggctaatgagattgaccatcagttctggaaggattttcgtaaggcagttttagctaaaaatcctgatctttatatcctaggagaagtctggcatacatctcagccttggctaaatggagatgagttccatgccgtcatgaattatcctttatctgatagtatcaaggactatttcttacgaggaattaagaagacagaccagttcatcgatgaaatcaatggagagtctatgtattacaagcagcagatttcagaggtcatgtttaatctcttggattcacatgatacagagcgaatcctgtggacggccaatgaagatgttcaactggttaaatcagccttagcctttctctttttacaaaaaggaacaccgtgcatttattacggaaccgagctagccttgactggaggaccagatccagattgtcgtcgttgtatgccttgggaacgtgtatcaagtgacaatgatatgctgaactttatgaagaggctgattaaaattcggaaatacgcgtcagtaatcatttcgcatggcaagtatagccttcaagaaatcaactctgatctagtagctctggaatggaaatacgaaggacggatcctcaaagcaatattcaaccaatcaacagaagattatcttttagagaaagaagcagtagcactagcaagcaattgccaagaattggataatcagcttgtcatctctccagatggatttatgattttctaa

SEQ ID NO: 30 - protein, 395 aa, Salmonella pneumoniae
MNYLQDLGITGLYLCPIFESTSNHKYNTTDYFEIDRHFGDKETFRELVDQAHHRGMKVMLDAVFNHIGSQSLQWKNVVKNGEQSAYKDWFHIQQFPVTTEKLVNKRDLPYHVFGFEDYMPKLNTANPEVKNYLLKVATYWIEEFNIDAWRLDVANEIDHQFWKDFRKAVLAKNPDLYILGEVWHTSQPWLNGDEFHAVMNYPLSDSIKDYFLRGIKKTDQFIDEINGESMYYKQQISEVMFNLLDSHDTERILWTANEDVQLVKSALAFLFLQKGTPCIYYGTELALTGGPDPDCRRCMPWERVSSDNDMLNFMKRLIKIRKYASVIISHGKYSLQEINSDLVALEWKYEGRILKAIFNQSTEDYLLEKEAVALASNCQELDNQLVISPDGFMIF

SEQ ID NO: 31 - DNA, 318 bp, Salmonella pneumoniae
  CDS (1)..(318)
atgaattgtagaggacatgaaacaagacaaagaattgttagagattttgaagttcagcctaaagcacatattaagctgttagcaaatcaacaaaaacatagtgatgcaggagcaactattgaagatgaatattatgtatttatcgctgagagtaaaattgatggcaagaaggaagttattcagtgttgcatgggtgcggcaagggattttttagaactaattaatcacaaagggctacctctttttaatccgcttgtaggtgattctcatgtaaataatagacaagaatatgacaatacagggagtggaaatttataa

SEQ ID NO: 32 - protein, 105 aa, Salmonella pneumoniae
MNCRGHETRQRIVRDFEVQPKAHIKLLANQQKHSDAGATIEDEYYVFIAESKIDGKKEVIQCCMGAARDFLELINHKGLPLFNPLVGDSHVNNRQEYDNTGSGNL

SEQ ID NO: 33 - DNA, 762 bp, Salmonella pneumoniae
  CDS (1)..(762)
atggaaatccaagattatactgatagtgaattcaaacatgctttagcaaggaatcttcgttcactgacaagaggaaaaaagtccagtaagcaacctatagcgattttgcttggagggcaaagtggtgccggtaagactacaattcatcgtattaaacagaaagaatttcaaggaaatattgttatcatagatggtgatagttttcgttctcagcatccacactatttagaactgcagcaagaatatggcaaagacagtgtagaatataccaaagattttgcaggaaaaatggtagagtctttagtaacaaaattgagtagtttgagatacaatcttttgatagagggaactttacgaacagttgatgttccaaagaaaacagcacaactcttgaaaaataagggatatgaagtacaattggccttaattgcgacaaagcctgaattgtcgtatctaagtactcttatccgttatgaagaactgtacattatcaatccaaatcaagcacgcgcaactccaaaagaacatcatgatttcattgtaaatcatctagttgataacacacgaaaattggaagaactagctatctttgaaagaattcaaatttaccaacgagatagaagttgtgtatatgattcaaaagaaaatacaacttcagcagcagatgttcttcaagagttactctttggggagtggagtcaggtagagaaggagatgttgcaggtgggggaaaagagacttaatgaattacttgaaaaataa

SEQ ID NO: 34 - protein, 253 aa, Salmonella pneumoniae
MEIQDYTDSEFKHALARNLRSLTRGKKSSKQPIAILLGGQSGAGKTTIHRIKQKEFQGNIVIIDGDSFRSQHPHYLELQQEYGKDSVEYTKDFAGKMVESLVTKLSSLRYNLLIEGTLRTVDVPKKTAQLLKNKGYEVQLALIATKPELSYLSTLIRYEELYIINPNQARATPKEHHDFIVNHLVDNTRKLEELAIFERIQIYQRDRSCVYDSKENTTSAADVLQELLFGEWSQVEKEMLQVGEKRLNELLEK

SEQ ID NO: 35 - DNA, 777 bp, Salmonella pneumoniae
  CDS (1)..(777)
atgagagggtttaataacaagataaagtctgtttatcaagaactaacaaattccaaagagaaattcggtagctttcacaagactttaattcatttgcatacacctgtttcttatgattacaagctattttctaattggactgcaacgaaatatagaaaaattactgaagatgaactatatgatatattttttgaaaataagaaaataaaagttgataagacaattttttttagtaattttgataaggttgttttttctagttcaaaagaatatattagttttcttatgttagcagaggcaatcataaaaaatggaatagaaatagttgtagtaactgatcataatactaccaaaggtattaaaaagttacaaatggcagtctcaatcataatgaaaaattatccgatttatgatatacatcctcatattttacatggagtagaaattagtgcagcagataaattgcatattgtatgtatatatgattatgaacaagaatcatgggttaatcaatggttaagtgaaaatattataagtgagaaagatggaagttatcaacattcactgactataatgaaggatttcaataatcaaaaaatagttaactatattgctcatttcaatagttatgacattttgaaaaaaggttctcacttatcaggtgcatataaacgaaaaattttttctaaagaaaatacacgattttggagtttaatattaactcgaaagaatcttcgcaacaacttgatattctctataaagaagttggtgtattaa

SEQ ID NO: 36 - protein, 258 aa, Salmonella pneumoniae
MRGFNNKIKSVYQELTNSKEKFGSFHKTLIHLHTPVSYDYKLFSNWTATKYRKITEDELYDIFFENKKIKVDKTIFFSNFDKVVFSSSKEYISFLMLAEAIIKNGIEIVVVTDHNTTKGIKKLQMAVSIIMKNYPIYDIHPHILHGVEISAADKLHIVCIYDYEQESWVNQWLSENIISEKDGSYQHSLTIMKDFNNQKIVNYIAHFNSYDILKKGSHLSGAYKRKIFSKENTRFWSLILTRKNLRNNLIFSIKKLVY

SEQ ID NO: 37 - DNA, 1824 bp, Salmonella pneumoniae
  CDS (1)..(1824)
atggtcatcactaaacactttgccattcacggaaagagttaccgcagaaagcttatcaagtacattctcaatcctgagaaaaccaataatcttgccttggtgtcggactatggcatgaagaattttctggactttcctagctatgaggaaatggtgcagatgtatcatgaaaatttcatcagcaacgatacgctttacgattttcgccacgacaggatggaagaaaatcaacgaaaaatacacgctcaccacatcattcagtctttctcgccagaggatcatatcactcctgaacaaatcaatcggataggttatgagactgtgaaggaattaactggtggcaaatttcgttttatcgttgcgacccatgttgataaagaccacctgcacaatcacatcattatcaattcagtagatagcaattctgacaaaaagctcaagtgggactacaaggtggagcgaaatcttcgcatgatttctgaccgtttttctaaaatcgcaggtgctaaaatcattgagaaccgctattctcaccagcggtatgaagtctatcgtaagactaatcacaagtatgaactcaagcagcgactctattttttgatggaacattctagggactttgaggatttcaaaaagaatgctccgctactacatgtggagatggatttccgtcacaagcatgccaccttttttattacggactcaactatgaaacaggtggtgcgtggcaagcaactcaatcgcaagcagccttacacagaagaattttttaagaactactttgccaaaagagaaatagaaagtctcatggaatttttattgctgaaagttgagaatatggatgatttacttcagaaagcaaaactttttggactaactatcaatcctaaacaaaagcatgtttcttttcaatttgcaggagtggaggtaaaggagacagagctagaccagaaaaatctttatgatgtagagtttttccaagattattttaaaaatagaaaagattggcaagctccagaaactgaggatttcgttcaactttatcaagaagaaaagttatccaaagaaaaagaacttccaagcgatgagaagttctgggagtcctatcaagagttcaagagtaacagagatgccgttcatgaatttgaggtggagttgtcactcaatcaaattgaaaaagtagtggatgatggaatttacgtcaaggtcaagtttggtattcgtcaggagggacttatctttgtgccgaacatgcagcttgatatggaagaggataaggtgaaggttttcatcagggaaaccagctcctactatgtctaccacaaagacgctgccgagaaaaattgttatatgaaaggtcgaaccttaattagacagttcagctatgaaaatcaaaccattccattacgcagaaaagcgacagtcgatatgattaaagagaagattgcggaagtggatgctttgattgaactggaagtagaaaatcaatcttatgtcacgattaaagatgagttagtgcatgaactagcagcgtctgaattgagaatcaatgagttgcaagaacgaatgtcaaccttgaatcaagtagcagaatatctactggcttcagttgaaagtaagcaagaaatgaaattaaatctttcaaaactgaatataactgagaatatcagtgctaatattgttgagaaaaaattgaagagcctggggaatcaactggaattggaaaggggcaggtatgaaaagatggtagtcaggctagataaatttataaatcgcttaaatacaggactttcaaaaggagatgggatagattttcaaaaatag

SEQ ID NO: 38 - protein, 607 aa, Salmonella pneumoniae
MVITKHFAIHGKSYRRKLIKYILNPEKTNNLALVSDYGMKNFLDFPSYEEMVQMYHENFISNDTLYDFRHDRMEENQRKIHAHHIIQSFSPEDHITPEQINRIGYETVKELTGGKFRFIVATHVDKDHLHNHIIINSVDSNSDKKLKWDYKVERNLRMISDRFSKIAGAKIIENRYSHQRYEVYRKTNHKYELKQRLYFLMEHSRDFEDFKKNAPLLHVEMDFRHKHATFFITDSTMKQVVRGKQLNRKQPYTEEFFKNYFAKREIESLMEFLLLKVENMDDLLQKAKLFGLTINPKQKHVSFQFAGVEVKETELDQKNLYDVEFFQDYFKNRKDWQAPETEDFVQLYQEEKLSKEKELPSDEKFWESYQEFKSNRDAVHEFEVELSLNQIEKVVDDGIYVKVKFGIRQEGLIFVPNMQLDMEEDKVKVFIRETSSYYVYHKDAAEKNCYMKGRTLIRQFSYENQTIPLRRKATVDMIKEKIAEVDALIELEVENQSYVTIKDELVHELAASELRINELQERMSTLNQVAEYLLASVESKQEMKLNLSKLNITENISANIVEKKLKSLGNQLELERGRYEKMVVRLDKFINRLNTGLSKGDGIDFQK

SEQ ID NO: 39 - DNA, 867 bp, Salmonella pneumoniae
  CDS (1)..(867)
gtgggtacactattagcaacaagattaaaaaatagacgaaaagaattaaaaatgtctcagcgagaattagctgagggtatctgtaaacaggggcaaattagtcgattagagagtggagagttcacccctggagcggattttttacatgcccttgctaagaaattaaaagttagtatggactacttttttgatgagcaggttgtagaggaagtcaatgagttatcagagtttaagaagttagcacagacatttatcacaaatcgtaattatgaatctttaaaatatatatatgaattggaaaatgtaaaagcccatcgcttatctctagcagataaattttacatggaatggataaagtctcttatagatttttatttttatggacagaaagaagaggcaattacacgattagagaatatgctacctcagttaaatgtatctgatttgaactaccttcaagtttcaaacactctattcaatttttattatgatattgaagatttagaacgttttaatgagattagagacaagctagagcatcaagtgaatcagcttaatttaaacacaattgaagagttagaactatcaattaagttcaattataacgtttgtcgatacctatggttacagaaaaatattgaagaagctattacgaaaatcacagctacgataaagcaatgtaaggagtacaggacaacttatctattagctgatttgtatttattgatgggaagtgtaagtgaaaatttttcttctaaaagttcagtaaaagaatattttgagactgctcattttctctacaaacttgaggaaaacatgtcaatggctttgaaagtcgaacattattttgcagatataacagaataa

SEQ ID NO: 40 - protein, 288 aa, Salmonella pneumoniae
VGTLLATRLKNRRKELKMSQRELAEGICKQGQISRLESGEFTPGADFLHALAKKLKVSMDYFFDEQVVEEVNELSEFKKLAQTFITNRNYESLKYIYELENVKAHRLSLADKFYMEWIKSLIDFYFYGQKEEAITRLENMLPQLNVSDLNYLQVSNTLFNFYYDIEDLERFNEIRDKLEHQVNQLNLNTIEELELSIKFNYNVCRYLWLQKNIEEAITKITATIKQCKEYRTTYLLADLYLLMGSVSENFSSKSSVKEYFETAHFLYKLEENMSMALKVEHYFADITE

SEQ ID NO: 41 - DNA, 1347 bp, Salmonella pneumoniae
  CDS (1)..(1347)
atgaattacgaaaagtatttaaactatttggactatgaaactgaaatagaagacgcataccataatttattattagaatataaaattagtgataattttagtgatgaacattggttatataatttgccgtctaatatcacccaatctaagggatttaaaatacacctgagcgcttcaatattaaatgctaatttggttgctaaaaaattctttgattttatattttctagagagaagaagataaatttcaaaattttggttagcattaaagaactttctttgcaaaatacagggcttaatggttattcacaagtcgggaaattcattacaatttatccaaaagataataaagagtttcaacgtttgttacacaaattagaaatattgtataaaggtgtaaagggtgtaaacataccatcagatttcagatttcaacttagcgaggttgtatattacagatatggggagttcgtaaaagatagcactttcaaggataaaagagataagaaaatcccttcaaatgtgaatgttccaataagggattactatattccaagatataacactattcctgatcaatatattattttggaagtgatttctaaaaatgccaaaggtggagtttataaagtatttaatactcaaaagcgagtttattctttattaaaagaagctagtgatttatcgttggttgattttacaaatagagattctgtgaatcgtttaataaatgaaagagaaatattagtagaattggaaaaggaggaatttactccaaaagtttttaacgatttttacataaagaattcgtattttgtggaatttgagtttattattgcagataaattaagcgagtataatatggttagtggctcgtatgattggtttttaacattaattgattatatggagataataaactccaaatataatctttcctatagagatttatcatttaataatatacttattgataatagaaataatatttatattatagactttgagcatgccctaaggaaagaaactcaaattgaagaaaaaaagttgcctctatttggaactccagggttttatgaaaccaacttaaatttattgaataatcaaccagaggatattatgggattggttttgttattatattggagtcagaatttagatgattttaacgtcttttcaaaaatgagttttttagaagcgatggagtatgcaatgaattttaattcgcgtcgcttagacacacttgataaatcagattggctttatgatatttataaaaaagcgttcaattataaatatgatactttttctgaattaaaaaaagattttatagaggtattaaaataa

SEQ ID NO: 42 - protein, 448 aa, Salmonella pneumoniae
MNYEKYLNYLDYETEIEDAYHNLLLEYKISDNFSDEHWLYNLPSNITQSKGFKIHLSASILNANLVAKKFFDFIFSREKKINFKILVSIKELSLQNTGLNGYSQVGKFITIYPKDNKEFQRLLHKLEILYKGVKGVNIPSDFRFQLSEVVYYRYGEFVKDSTFKDKRDKKIPSNVNVPIRDYYIPRYNTIPDQYIILEVISKNAKGGVYKVFNTQKRVYSLLKEASDLSLVDFTNRDSVNRLINEREILVELEKEEFTPKVFNDFYIKNSYFVEFEFIIADKLSEYNMVSGSYDWFLTLIDYMEIINSKYNLSYRDLSFNNILIDNRNNIYIIDFEHALRKETQIEEKKLPLFGTPGFYETNLNLLNNQPEDIMGLVLLLYWSQNLDDFNVFSKMSFLEAMEYAMNFNSRRLDTLDKSDWLYDIYKKAFNYKYDTFSELKKDFIEVLK

SEQ ID NO: 43 - DNA, 918 bp, Salmonella pneumoniae
  CDS (1)..(918)
atgaattgtttcttgaaaatgaataatgtaagtgttcgttatgatgacgtaatagctttaaaagatataactttacaaataaataagggagatttcattggcttattaggttcaaatggtgcaggtaaatctacgttaattaattctattgtaggttttcaagagatttatttaggagaaatagagtattgtgataaagatttgatagttagttctcaaccttttgctcatttaggctttactcctcaaaccacagtaattgatttttatactactgtgaaggacaatgtaatattggggctgaaccttgctggaaagtttgggaaaaatgctgagaagttgtgtcaaatagccttagaaattgttgggttagctgataaaaaaaataatttggtagaaacattgtcaggtggacaactgcaacgcgtccagattgctagagcaatagctcataatccagatttttatattttagatgaacctaccgttggtttagatactgaatctgccgaaaaatttttaatgtatttaaaagataagagtttggaaggaaaaactattatcatatcttcacatgacataaatctactcgaaaagttttgtaaaaaaatactttttttacaaaatggctccatatcattttttggtgatatgcgtgactttgtagataattcaactatcaaattaaatttttcaatgcagaatagaatttctagatatcaaattgaatttttagaaaattttagatttaaagttcacatcgaagataatgatagttttacaatagaagtccctatagaagaaaagatcttagatgttatcaatgaggtaggaaaagcatgtgaaattaaaaacttttcaacaagtaaattaaccttacaagaaagttatttgcaaagaataggaggagaaaaatga

SEQ ID NO: 44 - protein, 305 aa, Salmonella pneumoniae
MNCFLKMNNVSVRYDDVIALKDITLQINKGDFIGLLGSNGAGKSTLINSIVGFQEIYLGEIEYCDKDLIVSSQPFAHLGFTPQTTVIDFYTTVKDNVILGLNLAGKFGKNAEKLCQIALEIVGLADKKNNLVETLSGGQLQRVQIARAIAHNPDFYILDEPTVGLDTESAEKFLMYLKDKSLEGKTIIISSHDINLLEKFCKKILFLQNGSISFFGDMRDFVDNSTIKLNFSMQNRISRYQIEFLENFRFKVHIEDNDSFTIEVPIEEKILDVINEVGKACEIKNFSTSKLTLQESYLQRIGGEK

SEQ ID NO: 45 - DNA, 801 bp, Salmonella pneumoniae
  CDS (1)..(801)
atgaaggctgatcaattaaggcacaaatcggacttaggtttaagaggtctagcgattattgctaaaaatgagattattgctttttttagaagtaaaggtttaattatttctcagtttctacaaccaatcttatatgttgtttttataataataggattaaattcttcgataaagaacattcagtttaatgatataaaaacctcttatgcagaatatacaatcattggtgttatagctttattgataatcgggcagatgactcaagttatttatagggtgacaatagataaaaaatatgggctacttgctcttaagttatgcagtggagttcgtcctttatattatattttagggatgagtatctattctatattagggttgatagttcaagaaattattatatatataattacgttagcgtttgagataaatatcgcaatggatagatttttttatacagttttgttatctattgttgttttattattttgggactcccttgcaattttacttacaatgtttatcaatgattacagaagacgtgatattgtaatacgttttgtactaacaccgcttggttttacagctcctgttttctacttaatagattctgctcctagtattgtgagatggattggtcagttaaatcccttaacttatcaattaactattttgagaaacttttattttaaaaattcaacaactttggaattagttttcttattgttaacatcattacttgtccttatatctgtatcttttattataccaaagataaaattgatactgatagaaagataa

SEQ ID NO: 46 - protein, 266 aa, Salmonella pneumoniae
MKADQLRHKSDLGLRGLAIIAKNEIIAFFRSKGLIISQFLQPILYVVFIIIGLNSSIKNIQFNDIKTSYAEYTIIGVIALLIIGQMTQVIYRVTIDKKYGLLALKLCSGVRPLYYILGMSIYSILGLIVQEIIIYIITLAFEINIAMDRFFYTVLLSIVVLLFWDSLAILLTMFINDYRRRDIVIRFVLTPLGFTAPVFYLIDSAPSIVRWIGQLNPLTYQLTILRNFYFKNSTTLELVFLLLTSLLVLISVSFIIPKIKLILIER

SEQ ID NO: 47 - DNA, 210 bp, Salmonella pneumoniae
  CDS (1)..(210)
atgaaaattcattgtttaaaattaaaaaataaagaattgaataaagaagtagcattctatttaacttcaattattaggcaagctttaaaaaatactgaatataaagatcaaatatctagcacagtattgccagatatcaaaattaaattaccgatagacagtagaggtactccagattggaattacatggaaagatatagagatagataa

SEQ ID NO: 48 - protein, 69 aa, Salmonella pneumoniae
MKIHCLKLKNKELNKEVAFYLTSIIRQALKNTEYKDQISSTVLPDIKIKLPIDSRGTPDWNYMERYRDR

SEQ ID NO: 49 - DNA, 156 bp, Salmonella pneumoniae
  CDS (1)..(156)
atgataaataaactatccagatacatggaaagcagtggtaagacctatcaaaaccactatgtgacaatcttaaaatggtatgaagaagataaagacaaactaagacagaaaggtttaaataaaaaaatgaattatgatgtaggagaatctttatag

SEQ ID NO: 50 - protein, 51 aa, Salmonella pneumoniae
MINKLSRYMESSGKTYQNHYVTILKWYEEDKDKLRQKGLNKKMNYDVGESL

SEQ ID NO: 51 - DNA, 195 bp, Salmonella pneumoniae
  CDS (1)..(195)
atgaaaatggcaaccgataaaaatagaataatgattagcttggatgataaaaatctagagaaacttgaaaatttagtagaagatgctagagatagaaggggaatgcgactaacaaagtcccaggttattgaattactcctaaataccgttgattatttcgatgatattatgggggctatttactcaaaaaaataa

SEQ ID NO: 52 - protein, 64 aa, Salmonella pneumoniae
MKMATDKNRIMISLDDKNLEKLENLVEDARDRRGMRLTKSQVIELLLNTVDYFDDIMGAIYSKK

SEQ ID NO: 53 - DNA, 387 bp, Salmonella pneumoniae
  CDS (1)..(387)
atgaatactttaggagacaaattgatggaattgagtgctatttaccataggcctgagtcggagtatgactatctttataaggataagaaactccatattcgaattcgaactaagaaaggggacattgaaagcatcaacttgcactatggggacccttttatctttatggaggagttttatcaggatacaaaagaaatggtcaagataacttctggtaccttatttgaccattggcaggttgaagtgtcagttgactttgcacgtatccagtatctctttgagctcagagatacagaaggtcaaaatattttgtatggcgataaagggtgtgtggaaaattctctagaaaatcttcatgcaattgggaatggatttaagttgccttag

SEQ ID NO: 54 - protein, 128 aa, Salmonella pneumoniae
MNTLGDKLMELSAIYHRPESEYDYLYKDKKLHIRIRTKKGDIESINLHYGDPFIFMEEFYQDTKEMVKITSGTLFDHWQVEVSVDFARIQYLFELRDTEGQNILYGDKGCVENSLENLHAIGNGFKLP

SEQ ID NO: 55 - DNA, 282 bp, Salmonella pneumoniae
  CDS (1)..(282)
atgtggttgattattttatggaatgctaagccggatacacctttatttaattttaaagacgaagtaattaagtataaaacatatgagccttttgaaagcagtataaaaagagtaaatactactataaagaatggtagtaaagggaaaactctgactgagatgattaatggctacagagcggataacgatattagagatgaaatttgtaactttaatattctgaaaaataaaattcgtgatatgaaaaaccaacaaggaaatacaatggaatcttacttttag

SEQ ID NO: 56 - protein, 93 aa, Salmonella pneumoniae
MWLIILWNAKPDTPLFNFKDEVIKYKTYEPFESSIKRVNTTIKNGSKGKTLTEMINGYRADNDIRDEICNFNILKNKIRDMKNQQGNTMESYF

SEQ ID NO: 57 - DNA, 534 bp, Salmonella pneumoniae
  CDS (1)..(534)
ttggtgcgttatgcttttttatgctataatggatttataaaaataaaggagtttgctatgattggaaagaacataaaatccttgcgtaaaacacatgacttaacacaactcgaatttgcacggattgtaggtatttcacgaaatagtctgagtcgttatgaaaatggaacgagttcagtctctaccgaattaatagacatcatttgtcagaagtttaatgtatcttatgtcgatattgtaggagaagataaaatgctcaatcctgttgaagattatgaattgactttaaaaattgaaattgtgaaagaaagaggtgctaatctattatctcgactctatcgttatcaagatagtcagggaattagcattgatgatgagtctaatccttggattttaatgagtgatgatctatctgatttgattcatacgaatatctatctagtagaaacttttgatgaaatagagagatatagtggctatttggatggaattgaacgtatgttagagatatctgaaaaacggatggtggcctaa

SEQ ID NO: 58 - protein, 177 aa, Salmonella pneumoniae
LVRYAFLCYNGFIKIKEFAMIGKNIKSLRKTHDLTQLEFARIVGISRNSLSRYENGTSSVSTELIDIICQKFNVSYVDIVGEDKMLNPVEDYELTLKIEIVKERGANLLSRLYRYQDSQGISIDDESNPWILMSDDLSDLIHTNIYLVETFDEIERYSGYLDGIERMLEISEKRMVA

SEQ ID NO: 59 - DNA, 378 bp, Salmonella pneumoniae
  CDS (1)..(378)
ttgggacaaaaagttgtagccatgcttgattttttattagcatatagtgattattctaaagacttcagaccattgattattgatcagcctgaagacaatctagacaatcgttatatttacaggcatttagttcagcagtttagagatgtgaaagctcaacgtcaaattattttagcaacacataatgctacaattgtaacaaattctatgacagatcaagttgttattatggagtcagatggagttaacggatggattgaatcacagggatatgttagtgaaaaatatataaaaaatcatatcatcaatcaattagagggaggaaaagattccttcaagcataaaatgtctatatatgagacggctttatcagagtag

SEQ ID NO: 60 - protein, 125 aa, Salmonella pneumoniae
LGQKVVAMLDFLLAYSDYSKDFRPLIIDQPEDNLDNRYIYRHLVQQFRDVKAQRQIILATHNATIVTNSMTDQVVIMESDGVNGWIESQGYVSEKYIKNHIINQLEGGKDSFKHKMSIYETALSE

SEQ ID NO: 61 - DNA, 393 bp, Salmonella pneumoniae
  CDS (1)..(393)
ttgtcagtacaagtattttggaaaggaagtagcagtatgaaacgagatgtgcgtgatattcggaaacaatttcgtttaacagaagcagaagaaaagcaaattctagctttgatgagagagcggggagagactaatttctctgattttcttcgtaaaagtttactttcctctgatttacaaaaacagatggagacatggtttgccctctggcaatcccaaaaactagaacaaatcagtcgtgacgttcatgaagttttaatcttggcacagtcagaacgtcaagtcacccaagagcatgtatctattctcttaacgtgcgtgcaggaattgattcaagaggttgcaaacaccatacccctcagtaaagaatttcgtgagaagtacatgaggtaa

SEQ ID NO: 62 - protein, 130 aa, Salmonella pneumoniae
LSVQVFWKGSSSMKRDVRDIRKQFRLTEAEEKQILALMRERGETNFSDFLRKSLLSSDLQKQMETWFALWQSQKLEQISRDVHEVLILAQSERQVTQEHVSILLTCVQELIQEVANTIPLSKEFREKYMR

SEQ ID NO: 63 - DNA, 366 bp, Salmonella pneumoniae
  CDS (1)..(366)
atggaacatcgttaccgaaccaatctcaagaaagtgtttttgtctgatagtgagttgaaccaactaaatataaatatcgatcaaagtggttgtaaatccttttctgaatatgcgagacgaactctactcgatcctggtatgaattttatcacgattgacacaaacggttaccaagatttagtgtttgagttaaagaggattggcaataatatcaaccagattgctcgaagtgttaatcaatctcagttaatttctggtgaagaattgcaggagttgaaaaaaggaattggtgaattgataaaagaagttgataaggaatttaatctgcaagcgcagaagctaaaggagttccatggtcatcactaa

SEQ ID NO: 64 - protein, 121 aa, Salmonella pneumoniae
MEHRYRTNLKKVFLSDSELNQLNINIDQSGCKSFSEYARRTLLDPGMNFITIDTNGYQDLVFELKRIGNNINQIARSVNQSQLISGEELQELKKGIGELIKEVDKEFNLQAQKLKEFHGHH

SEQ ID NO: 65 - DNA, 252 bp, Salmonella pneumoniae
  CDS (1)..(252)
atgagtagtcaaatgaaagctttcttgaatgaacttcaaggaaatatggaagttgttggagaattttttgataatcctgaaaaggtaattagaaaatttggaattgtcggaagagagaaggaagcattgctaatccgtgacttaaatgatatggatgattatgctctttcaattcaaaatagcgttgcatcgccttctggggcacatagtagcacttgccattttgttagacaagatttacaatccgcataa

SEQ ID NO: 66 - protein, 83 aa, Salmonella pneumoniae
MSSQMKAFLNELQGNMEVVGEFFDNPEKVIRKFGIVGREKEALLIRDLNDMDDYALSIQNSVASPSGAHSSTCHFVRQDLQSA

SEQ ID NO: 67 - DNA, 303 bp, Salmonella pneumoniae
  CDS (1)..(303)
atgaaaaataaatacgatgttaaaagaattattccggatgaattatcagaatcactagatatttttctaaaaaattattctgaaactggattgtcagattataatacttatctcttttatggttttatcttgaagtcctataaattgcctcgagaaaatagatatagcataaaattattggtgaaggaattacaaaatagaggactgaaagttacattgattatcaacatttattatcacgctttaaattgtttggctttaaatgatggtctaaaaatttatggagaagatttcttaatatga

SEQ ID NO: 68 - protein, 100 aa, Salmonella pneumoniae
MKNKYDVKRIIPDELSESLDIFLKNYSETGLSDYNTYLFYGFILKSYKLPRENRYSIKLLVKELQNRGLKVTLIINIYYHALNCLALNDGLKIYGEDFLI

SEQ ID NO: 69 - DNA, 1017 bp, Salmonella pneumoniae
  CDS (1)..(1017)
atgaaaaagaaatggatgtattatgctgcttgttcttctaatgaatctgccgatgacagttcatctgataaaggagacggcggttcgctagtcgtttattcaccaaactcagagggcttaattggagcaactattcctgcctttgaagaaaaatatggtatcaaagtagaactgattcaagctggtactggagaacttttcaaaaaactagagtcagaaaaagaagttcctgtagctgatgttatctttggtggttcttatacacaatatactacccacggagaactctttgaaaactatacttcaaaagaaaatgataatgttatcaaagaatatcaaaacacaactggctactctactccttatacactagatggtagtgttttaatcgtcaaccctgatttaactaaaggcatgaacatcgaaggatataacgatcttttcaaacctgaactaaaaggaaaaatcgcaactgctgacccagcaaactcttctagcgcctttgctcaattaacaaatatgctacaagctcaaggtggttacaaagatgataaggcttggtcttatgtaaaagatcttttcacacttattgatggtaaaatcggttcaagttcatctagtgtctataaagtagtcgctgatggagaaatggctgttggtctctcttatgaagatccagcagttaaactcttaaatgacggagctaacattaaggtagtctatccaaaagaaggaaccgtcttcctacctgctagtgctgctatcgttaaaaaatctaaaaatatggaaaatgccaagaaatttatcgattttattatctctcaagaagtacaagatacacttggtacaaccactactaaccgtcctgttcgtaaaaatgctaaaacaagcgaaaacatgaaaccaattgacaaaatcaaaacactcactgaagattatgattatgtcatcaagaataaatcagatatcgttaagaaatacaacgaagtctttacagatatccaatctaaacagtaa

SEQ ID NO: 70 - protein, 338 aa, Salmonella pneumoniae
MKKKWMYYAACSSNESADDSSSDKGDGGSLVVYSPNSEGLIGATIPAFEEKYGIKVELIQAGTGELFKKLESEKEVPVADVIFGGSYTQYTTHGELFENYTSKENDNVIKEYQNTTGYSTPYTLDGSVLIVNPDLTKGMNIEGYNDLFKPELKGKIATADPANSSSAFAQLTNMLQAQGGYKDDKAWSYVKDLFTLIDGKIGSSSSSVYKVVADGEMAVGLSYEDPAVKLLNDGANIKVVYPKEGTVFLPASAAIVKKSKNMENAKKFIDFIISQEVQDTLGTTTTNRPVRKNAKTSENMKPIDKIKTLTEDYDYVIKNKSDIVKKYNEVFTDIQSKQ

SEQ ID NO: 71 - DNA, 1092 bp, Salmonella pneumoniae
  CDS (1)..(1092)
atgagtgagatcaaaattattaacgccaaaaaaatctaccacgatgtccctgttattgagaatttgaacattacaattccaaaaggaagtctctttacccttcttggagcttcaggatgtgggaaaacgacccttcttcgtatgattgcaggtttcaacagtatcgaaggtggagaattttacttcgatgatacaaaaatcaataatatggaacccagcaaacgcaatatcgggatggttttccaaaactacgctattttcccacatttgactgtccgagacaacgttgcttttggtcttatgcaaaagaaggttccaaaagaagaattgattcaacagaccaacaagtatcttgaactcatgcaaattgctcaatatgcggatcgaaagcccgataaactcagtggtggacaacaacaacgtgtcaccttggcatgcgccttagcggttaatccaagtgttctcctcatggacgagccacttagtaatctggaggccaaacttcgcttggatatgcgtcaagccatccgagaaatccaacacgaactgggaattacaactgtttatgtaacccacgaccaagaagaagccatggctatttcagaccaaattgctgttatgaaagatggggtgatccaacaaatcggcgcaccaaaagaactctatcataaaccagctaatgagtttgtggcaacctttatcggacgcacaaatattatccctgccaatcttgaaaaacggagcgacggcgcttatatcgtcttttcagatggctatgcccttcgaatgccagctcttgatcaggttgaggagcaagctattcatgtaagcattcgtcccgaagagtttatcaaagatgaatctggagatattgaaggaactattagagatagcgtctatcttggactaaatacggattatttcattgagacaggttttgcctcaaaaattcaagttagtgaagaatcaacttttgaagaagatctacaaaaaggcaatcgtattcgtctacgaatcaatacgcaaaaattaaacatcttttctgcagatggttcccaaaacctgataaaaggagtcaaccatggaacgtaa

SEQ ID NO: 72 - protein, 363 aa, Salmonella pneumoniae
MSEIKIINAKKIYHDVPVIENLNITIPKGSLFTLLGASGCGKTTLLRMIAGFNSIEGGEFYFDDTKINNMEPSKRNIGMVFQNYAIFPHLTVRDNVAFGLMQKKVPKEELIQQTNKYLELMQIAQYADRKPDKLSGGQQQRVTLACALAVNPSVLLMDEPLSNLEAKLRLDMRQAIREIQHELGITTVYVTHDQEEAMAISDQIAVMKDGVIQQIGAPKELYHKPANEFVATFIGRTNIIPANLEKRSDGAYIVFSDGYALRMPALDQVEEQAIHVSIRPEEFIKDESGDIEGTIRDSVYLGLNTDYFIETGFASKIQVSEESTFEEDLQKGNRIRLRINTQKLNIFSADGSQNLIKGVNHGT

SEQ ID NO: 73 - DNA, 423 bp, Salmonella pneumoniae
  CDS (1)..(423)
atggaacgtaaaaaactaaatatttggacagtttcctctttcttcctctttcttacctatcctatctttctcgtttatcctatcgttaccgtgctcaagcaagcacttatacatgaaggacaattctcactagctaattttgtcactttctttagtaaagcctactactctgagacactagtcaacagtttcaaggtttccattaccgctactatcacttccttagttgtaggaaccctattagcttatctcttctctatgtatgacttcaaggggaagaaatttctacaaatattgattatcattgcttccatgtcagctcctttcgtaggagcctactcctgggttctcttgctaggacgaaatgaggtcatcactaaatttttgacaaatgccctttatcttccagctatcgatatttat

SEQ ID NO: 74 - protein, 141 aa, Salmonella pneumoniae
MERKKLNIWTVSSFFLFLTYPIFLVYPIVTVLKQALIHEGQFSLANFVTFFSKAYYSETLVNSFKVSITATITSLVVGTLLAYLFSMYDFKGKKFLQILIIIASMSAPFVGAYSWVLLLGRNEVITKFLTNALYLPAIDIY

SEQ ID NO: 75 - DNA, 824 bp, Salmonella pneumoniae
  CDS (1)..(822)
atgagagcattctcagactttggaacgcctatgttgattggcgaagggtatcggactttccctgtcctgatttatacccaatttattagcgaggttggaggaaattctgcttttgcaattatggcgattatcattgccttggcaattttccttatccaaaaacacattgcaaaccgctacagtttcagcatgaatctgctccatccaattgagcctaaaaaaactacaaaaggaaaaatggctgccatttatgcaacagtctacggaattatctttatctctgttttacctcaaatctacttaatttatacctctttcctaaaaacatcagttatggtatctgttaaaggttattctccaaacagttacaaggtagctttccatcgtatgggatctgctattttcaataccattcgtatccctttgattgccttagttctagttgttctatttgcgacatttatctcctacctagccgttagaaaacggaatttgtttacaaacttaattgacagcctcagtatggtaccttatattgtaccaggaaccgttctagggattgccttcatttcttccttcaatactggtctatttggaagtggatttcttatgattacagggactgctttcatcttgattatgtctctatctgccagaagattaccttatactattcgctcatctgttgctagcttacaacaaatagcaccaagtattgaagaagctgctgaaagcttaggaagtagtcgtctcaatacctttgctaagattacaactccaatgatgctatctggtatcatttctggagccatcttatcttgagt

SEQ ID NO: 76 - protein, 273 aa, Salmonella pneumoniae
MRAFSDFGTPMLIGEGYRTFPVLIYTQFISEVGGNSAFAIMAIIIALAIFLIQKHIANRYSFSMNLLHPIEPKKTTKGKMAAIYATVYGIIFISVLPQIYLIYTSFLKTSVMVSVKGYSPNSYKVAFHRMGSAIFNTIRIPLIALVLVVLFATFISYLAVRKRNLFTNLIDSLSMVPYIVPGTVLGIAFISSFNTGLFGSGFLMITGTAFILIMSLSARRLPYTIRSSVASLQQIAPSIEEAAESLGSSRLNTFAKITTPMMLSGIISGAILS

SEQ ID NO: 77 - DNA, 26 bp, Salmonella pneumoniae
gctctagacccacaagatgctcttcg

SEQ ID NO: 78 - DNA, 29 bp, Salmonella pneumoniae
cgctctagacgcttgtcgttagcgccacc

SEQ ID NO: 79 - DNA, 24 bp, Salmonella pneumoniae
ggggtacccacaagatgctcttcg

SEQ ID NO: 80 - DNA, 28 bp, Salmonella pneumoniae
cggggtaccgcttgtcgttagcgccacc

SEQ ID NO: 81 - DNA, 27 bp, Salmonella pneumoniae
gctctagaagctatcgccgcccttgag

SEQ ID NO: 82 - DNA, 29 bp, Salmonella pneumoniae
cgctctagagcaacctgcggctagtttcc

SEQ ID NO: 83 - DNA, 28 bp, Salmonella pneumoniae
gctctagagttttagatcatgctttcgg

SEQ ID NO: 84 - DNA, 30 bp, Salmonella pneumoniae
cgctctagatttgtatgctgctacaggagc

SEQ ID NO: 85 - DNA, 27 bp, Salmonella pneumoniae
gctctagattgggtcaaatggttgtgg

SEQ ID NO: 86 - DNA, 27 bp, Salmonella pneumoniae
cgctctagaactagtcgttgtactttc

SEQ ID NO: 87 - DNA, 18 bp, Salmonella pneumoniae
cacaccgtaatcaagatc

SEQ ID NO: 88 - DNA, 18 bp, Salmonella pneumoniae
ctggtccgtaatatagtc

SEQ ID NO: 89 - DNA, 18 bp, Salmonella pneumoniae
ggtctattcgaccaccag

SEQ ID NO: 90 - DNA, 18 bp, Salmonella pneumoniae
ctggtgacctgcatcagc

SEQ ID NO: 91 - DNA, 16 bp, Salmonella pneumoniae
cacgagtatctacgtc

(19) United States
(12) Patent Application Publication
(10) Pub. No.: US 2004/0116661 A1
(43) Pub. Date: Jun. 17, 2004

(54) Streptococcal genes

(76) Inventors: David William Holden, London (GB); Jeremy Stuart Brown, London (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/415,478

(22) PCT Filed: Oct. 26, 2001
(86) PCT No.: PCT/GB01/04749

(30) Foreign Application Priority Data
Oct. 26, 2000 (GB) 0026231.1
Nov. 21, 2000 (GB) 0028345.7
Feb. 2, 2001 (GB) 0102666.5

(51) Int. Cl. C12Q 1/68; C07K 1/00; C07K 14/00; C07K 17/00

(52) U.S. Cl. 530/350

(57) ABSTRACT

Peptides derived from S. pneumoniae are identified as virulence determinants and may be useful in the preparation of vaccines for the treatment of infection. The peptides may be used as antigens or in the preparation of attenuated microorganisms for use as live oral vaccines.

### FIELD OF THE INVENTION

This invention relates to genes and the proteins that they encode, to vaccines containing the proteins or functional fragments of the proteins, and to live attenuated bacterial vaccines lacking any or part of the genes. More particularly, the invention relates to their prophylactic and therapeutic uses and their use in diagnosis.

### BACKGROUND OF THE INVENTION

High affinity iron uptake mechanisms are essential for the virulence of several Gram-negative pathogens, including pathogenic Neisseria (Schryvers and Stojiljkovic, 1999), Salmonella typhimurium (Janakiraman and Slauch, 2000), Pseudomonas aeruginosa (Takase et al., 2000), Legionella pneumophila (Viswanathan et al., 2000) and Yersinia pestis (Bearden and Perry, 1999). The mechanisms by which Gram-negative pathogens obtain iron from a host have been well described. They include the secretion of low molecular weight iron chelators, called siderophores, which scavenge iron from host iron-binding proteins such as transferrin, and secreted haemophores which acquire iron from haemoglobin and haemin (Wooldridge and Williams, 1993; Wandersman and Stojiljkovic, 2000). Alternatively, proteins containing iron or haem may bind to specific receptors on the bacterial outer membrane (Wooldridge and Williams, 1993; Cornelissen and Sparling, 1994). Independent of the source of the captured iron, transport into the bacterial cytoplasm is usually dependent on cytoplasmic membrane ABC transporters (Fetherston et al., 1999).

Despite the wealth of data on iron uptake by Gram-negative pathogens, little is known about the mechanisms and importance during infection of iron acquisition by Gram-positive pathogens. Of the Gram-positive pathogens, Streptococcus pneumoniae is second only to M. tuberculosis as a cause of mortality worldwide. S. pneumoniae frequently colonises the nasopharynx and invasive infection can develop in a variety of body compartments, including the middle ear, the lung interstitium, the blood, and cerebrospinal fluid. The organism must utilise iron sources in each of these environments, but at present it is poorly understood how S. pneumoniae acquires iron and from which substrate(s). Potential iron sources in the respiratory tract include lactoferrin, transferrin, ferritin (released from dead cells shed from the mucosal epithelium), and possibly small amounts of haemoglobin and its breakdown products (LaForce et al., 1986; Thompson et al., 1989; Schryvers and Stojiljkovic, 1999). In addition, siderophores produced by other nasopharyngeal commensuals may provide an alternative iron source. S. pneumoniae growth in iron-deficient medium can be complemented by haemin, haemoglobin and ferric sulphate but, in contrast to other pathogens of mucosal surfaces (Schryvers and Stojiljkovic, 1999; Cornelissen and Sparling, 1994), not by transferrin and lactoferrin (Tai et al., 1993). Chemical and biological assays suggest S. pneumoniae does not produce siderophores (Tai et al., 1993), but a haemin-binding polypeptide has been isolated and an undefined mutant unable to utilise haemin as an iron source was reduced in virulence (Tai et al., 1993 and 1997). However, the molecular basis for iron uptake by S. pneumoniae has yet to be characterised, and iron transporters have not been proven to be virulence determinants in animal models for a Gram-positive pathogen.

Virulence determinants of Gram-negative bacteria, including iron and magnesium transporters, are frequently encoded in defined areas of chromosomal DNA thought to be acquired by horizontal transmission and termed pathogenicity islands (PIs) (Carniel et al., 1996; Hacker et al., 1997; Blanc-Potard and Groisman, 1997; Janakiraman and Slauch, 2000). Characteristically, PIs have a different GC content to host chromosomal DNA, frequently have tRNA or insertion sequences at their boundaries, contain genes encoding mobile genetic elements, and are not present in less pathogenic but related strains of bacteria (Hacker et al., 1997). PIs often contain genes encoding virulence functions specific for the host bacteria. For example, SPI-2 of S. typhimurium encodes a type III secretion apparatus which allows the bacteria to multiply within macrophages but is not present in closely related enteric pathogens (Shea et al., 1996). Consequently, acquisition of PIs is probably a major influence in the evolution of distinct Gram-negative pathogens (Hacker et al. 1997; Ochman et al, 2000). In contrast, only a few PIs have been described for Gram-positive pathogens and they rarely have the classical genetic characteristics of Gram-negative PIs (Hacker et al., 1997). Those PIs of Gram-positive pathogens which have similar genetic characteristics to Gram-negative PIs encode toxins and are not required for the in vivo growth of the pathogen (Braun et al., 1997; Lindsay et al., 1998). S. pneumoniae is naturally transformable and readily integrates partially homologous DNA from other streptococci into its chromosome (Claverys et al., 2000). However, this mechanism of horizontal transfer of DNA is distinct from the acquisition of PIs and no S. pneumoniae PIs have been described.

It is desirable to provide means for prevention and therapy of Gram-positive bacterial infections and S. pneumoniae infections.

### SUMMARY OF THE INVENTION

Signature-tagged mutagenesis and genome searches have been used to identify three separate S. pneumoniae iron uptake ABC transporters, called Sit1, Sit2 and Sit3. The sequences of the Sit1, Sit2 and Sit3 ABCD genes are shown in the accompanying sequence listing. The Sit2 operon is located in a pathogenicity island required for in vivo growth. Other genes that encode putative virulence determinants were also identified, and are referred to herein as MS1 to 11 and ORF1 to 14.

According to a first aspect of the invention, a peptide is encoded by any of the gene sequences identified herein as Sit1A, B or C, Sit2B, C or D, Sit3A, B, C or D, ORF1 to 14, and MS1 to 11, or a functional fragment thereof, for therapeutic or diagnostic use.

According to a second aspect, an attenuated microorganism comprises a mutation that disrupts expression of any of the gene sequences identified above and also Sit1D and Sit2A.

According to a third aspect of the invention, a vaccine composition comprises any of the gene sequences identified above, with an optional pharmaceutically acceptable diluent, carrier or adjuvant.

In a specific embodiment, a vaccine composition comprises a peptide encoded by Sit1D and a peptide encoded by Sit2A, or a functional fragment thereof capable of elliciting an immune response. This combination vaccine ellicits a very good immune response compared to other known peptide vaccines.

According to a fourth aspect of the invention, a peptide of the invention is used in a screening assay for the identification of an antimicrobial drug, or in a diagnostic assay for the detection of a streptococcal microorganism.

### DESCRIPTION OF THE FIGURES

The invention is described with reference to the accompanying figures, wherein:

FIG. 1 is a schematic representation of the Sit1 and Sit2 loci, where clear boxes represent ORFs flanking the Sit loci; black boxes represent putative iron-binding receptors; grey boxes represent putative ATPases; diagonal shading represent putative permeases; and arrows represent site of insertions in mutant strains;

FIG. 2 is a schematic representation of PPI1, where (A) and (B) show the GC content plot for PPI1, with the dashed line representing mean GC content of S. pneumoniae DNA (38.9%), and (C) shows ORFs flanking and contained within PPI1, where ORFs present in a species are marked by a cross and ORFs absent from a species are marked by a minus mark;

FIG. 3 is a graphic representation of the growth of sit mutants measured by optical density, where (A) shows growth in THY broth, (B) in Chelex-THY broth, (C) in Chelex-THY broth+10 μM FeCl₂, (D) in Chelex-THY broth+10 μM FeCl₃ and (E) in Chelex-THY broth+10 μM haemoglobin, and where ⋄ represent the wild-type strain; □ a Sit1A⁻ strain; ◯ a Sit2A⁻ strain; ▴ a Sit1A⁻/Sit2A⁻ strain and where (F) shows the growth of the Sit1A⁻/Sit2A⁻ strain in Chelex-RPMlm with X representing no supplement; ▪ represents 10 μM FeCl₃; ◯ represents 10 μM haemoglobin; Δ represents 25 μM MnSO₄ and 25 μM ZnSO₄ supplements;

FIG. 4 illustrates the sensitivity to streptonigrin of the Sit⁻ strains;

FIG. 5 is a graphic representation of ⁵⁵FeCl₃ uptake, where

(A) represents the wild-type, Sit1A⁻, Sit2A⁻, and Sit1A⁻/Sit2A strains after 15 minutes, and (B) represents the wild-type and Sit1A⁻/Sit2A strains after 15 and 30 minutes;

FIG. 6 is a graphic representation of the survival of groups of 10 mice inoculated with Sit⁻ mutant strains where (A) represents intranasal (IN) inoculation and (B) represents intraperitoneal (IP) inoculation,

FIG. 7 is a graphic representation of the % survival of mice treated with various proteins and infected with S. pneumoniae; and

FIG. 8 is a graph showing the survival rates for mice which were administered serum from mice immunised with various proteins of the invention, wherein series 1 represents alum only, series 2 is Sit1D, series 3 is Sit2A, series 4 is pdb, series 5 is pdb+Sit1D, series 6 is pdb+Sit2A and series 7 is Sit1D+Sit2A.

### DESCRIPTION OF THE INVENTION

The peptides (proteins) and genes of the invention were identified as putative virulence determinants using signature tagged mutagenesis (Hensel et al., 1995).

The proteins and genes of the present invention may be suitable candidates for the production of therapeutically-effective vaccines against Gram-positive bacterial pathogens and S. pneumoniae. The term “therapeutically-effective” is intended to include the prophylactic effect of the vaccines. For example, a recombinant protein may be used, as an antigen for direct administration to an individual. The protein may be isolated directly from a Gram-positive bacterial pathogen or from S. pneumoniae or expressed in any suitable expression system, e.g. Escherishia coli. It is preferably administered with an adjuvant, e.g. alum.

In a preferred embodiment, the vaccine composition comprises a combination of peptides of the invention, e.g. a peptide encoded by Sit1D and a peptide encoded by Sit2A, or a functional fragment thereof capable of elliciting an immune response. This double protein vaccine is shown to offer improved protection compared to the known protective antigen non-toxic pneumolysin (Pdb).

The protein may be a mutant protein in comparison to wild-type protein, a fragment of the protein or a chimeric protein comprising different fragments or proteins, provided an effective immune response is generated. Preferably, protein fragments are at least 20 amino acids in size, more preferably, at least 30 amino acids and most preferably, at least 50 amino acids in size.

An alternative approach is to use a live attenuated Gram-positive bacterium or a live attenuated S. pneumoniae vaccine. This may be produced by deleting or disrupting the expression of a gene of the invention. Preferably, the S. pneumoniae strain comprises additional virulence gene mutations.

The mutated microorganisms of the invention may be prepared by known techniques, e.g. by deletion mutagenesis or insertional inactivation of a gene of the invention. The gene does not necessarily have to be mutated, provided that the expression of its product is in some way disrupted. For example, a mutation may be made upstream of the gene, or to the gene regulatory systems. The preparation of mutant microorganisms having a deletion mutation are shown in WO-A-96/17951. In one suitable technique, a suicide plasmid comprising a mutated gene and a selective marker is introduced into a microorganism by conjugation. The wild-type gene is replaced with the mutated gene via homologous recombination, and the mutated microorganism is identified using the selective marker.

The attenuated microorganisms may be used as carriers of heterologous antigens or therapeutic proteins/polynucleotides. For example, a vector expressing an antigen may be inserted into the attenuated strain for delivery to a patient. Conventional techniques may be used to carry out this embodiment.

Suitable heterologous antigens will be apparent to the skilled person, and include any bacterial, viral or fungal antigens and allergens, e.g. tumour-associated antigens. For example, suitable viral antigens include: hepatitis A, B and C antigens, herpes simplex virus HSV, human papilloma virus HPV, respiratory syncytial virus RSV, (human and bovine), rotavirus, norwalk, HIV, and varicella zooster virus (shingles and chickenpox). Suitable bacterial antigens include those from: ETEC, Shigella, Campylobacter, Helicobacter, Vibrio cholera, EPEC, EAEC, Staphylococcus aureus toxin, Chlamydia, Mycobacterium tuberculosis, Plasmodium falciparum, Malaria and Pseudomonas spp.

The heterologous antigen may be expressed in the host cell utilising a eukaryotic DNA expression cassette, delivered by the mutant. Alternatively, the heterologous antigen may be expressed by the mutant bacterium utilising a prokaryotic expression cassette.

The microorganism may alternatively be used to deliver a therapeutic heterologous peptide or polynucleotide to a host cell. For example, cytokines are suitable therapeutic peptides (proteins), which may be delivered by the microorganisms for the treatment of patients infected with hepatitis. The delivery of a polynucleotide is desirable for gene therapy, for example, anti-sense nucleotides, such as anti-sense RNA, or catalytic RNA, such as ribozymes.

Methods for preparing the microorganisms with the heterologous antigens etc, will be apparent to the skilled person and are disclosed in Pasetti et al., Clin. Immunol, 1999; 92(1): 76-89, which is incorporated herein by reference.

The gene that encodes the heterologous product may be provided on a recombinant polynucleotide that contains the regulatory apparatus necessary for the expression of the gene, e.g. promoter, enhancers etc. For example, the prokaryotic or eukaryotic expression cassette may be incorporated in a vector, e.g. a multi-copy plasmid. Alternatively, the heterologous gene may be targeted to a gene endogenous to the microorganism, including the gene to be mutated, so that the heterologous gene becomes incorporated into the genome of the microorganism, and uses the endogenous or cloned regulatory apparatus for its expression.

The protein (or fragments thereof) of the present invention may also be used in the production of monoclonal and polyclonal antibodies for use in passive immunisation.

In a further embodiment of the invention, the protein or corresponding polynucleotide may be used as a target for screening potentially useful drugs, especially antimicrobials. Suitable drugs may be selected for their ability to bind to the protein or DNA to exert their effects. Suitable drugs may be selected for their ability to affect the expression of Gram-positive pathogenicity island genes required for in vivo growth, for example the Sit genes, thereby reducing or altering the ability of the bacterium to survive in vivo or in a particular environment. Assays for screening for suitable drugs and which make use of the protein or polynucleotides of the invention will be apparent to those skilled in the art.

Although the proteins, polynucleotides, attenuated mutants and antibodies raised against the proteins and attenuated mutants are described for use in the diagnosis or treatment of individuals, veterinary uses are also considered to be within the scope of the present invention.

In a further embodiment, promoter sequences associated with the genes identified herein may be used to regulate expression of heterologous genes. This may be achieved either by incorporating the promoters in a vector system, e.g. a conventional gene expression vector. Alternatively, the heterologous gene may be inserted into the bacterial chromosome such that the promoter regulates expression.

In general, the techniques required to carry out the invention are those known conventionally in the art. Particular guidance is given in Sambrook et al, Molecular Cloning, A Laboratory Manual (1989), and Ausubel et al, Current Protocols in Molecular Biology (1995), John Wiley & Sons Inc.

The present invention relates to pathogenicity islands, containing genes required for the growth of Gram-positive pathogens in vivo. The example is described with reference to S. pneumoniae strain 0100993, however all pathogenic S. pneumoniae strains should have the same genes. Southern analysis and PCR have been utilised to demonstrate that Sit1 and Sit2 homologues are present in all S. pneumoniae capsular types tested. Sit1 probes hybridise to genomic fragments of other Streptococci e.g. S. mitis, S. oralis, S. sanguis, S. milleri. Vaccines to each of these may be developed in the same way as described for S. pneumoniae.

To formulate the vaccine compositions, the mutant microorganisms may be present in a composition together with any suitable excipient. For example, the compositions may comprise any suitable adjuvant. Alternatively, the microorganisms may be produced to express an adjuvant endogenously. Suitable formulations will be apparent to the skilled person. The formulations may be developed for any suitable means of administration. Preferred administration is via the oral, mucosal (e.g. nasal) or systemic routes.

Preferably, the peptides that may be useful for the production of vaccines have greater than 40% similarity with the peptides identified herein. More preferably, the peptides have greater than 60% sequence similarity. Most preferably the peptides have greater than 80% sequence similarity, e.g. 95% similarity. Preferably, the nucleotide sequences that may be useful for the production of vaccines have greater than 40% identity with the nucleotide sequences identified herein. More preferably, the nucleotide sequences have greater than 60% sequence identity. Most preferably the nucleotide sequences have greater than 80% sequence identity, e.g. 95% identity.

“Similarity” and “identity” are known in the art. In the art, identity refers to the relatedness between polynucleotide or polypeptide sequences as determined by comparing the sequences, and particularly identical matches between nucleotides or amino acids in correspondingly identical positions in the sequences being compared. Similarity refers to the relatedness of polypeptide sequences, and takes account not only of identical amino acids in corresponding positions, but also functionally similar amino acids in corresponding positions. Thus similarity between polypeptide sequences indicates functional similarity, even if there is little apparent identity.

Levels of identity between genes and levels of identity and similarity between proteins can be calculated using known methods. In relation to the present invention, publicly available computer based methods for determining identity and similarity between polypeptide sequences and identity between polynucleotide sequences include but are not limited to those of the GCG package (Genetics Computer Group, (1991), Program Manual for the GCG Package, Version 7, April 1991, 575 Science Drive, Madison, Wis., USA 53711), BLASTP, BLASTN, and FASTA (Atschul, S. F. et al., J. Molec. Biol. 215: 403-410 (1990)). The BLASTX program is available from NCBI and other sources. The Smith Waterman algorithm may also be used to determine identity. The parameters for polypeptide sequence comparison include the following:

Algorithm: Needleman and Wunsch (J. Mol Biol. 48: 443-453 (1970)).

Comparison matrix: BLOSSUM62 (Hentikoff and Hentikoff, PNAS 89: 10915-10919 (1992)).

Gap penalty: 12

Gap length penalty: 4

These parameters are the default parameters of the “Gap” program from Genetics Computer Group, Madison Wis.

The parameters for polynucleotide sequence comparison include the following:

Algorithm: Needleman and Wunsch (J. Mol Biol. 48: 443-453 (1970)).

Comparison matrix: matches=+10, mismatch=0

Gap penalty: 50

Gap length penalty: 3 available as the “Gap” program from Genetics Computer Group, Madison Wis.

These parameters are the default parameters for nucleic acid comparisons.

Table 1 lists the genes of the present invention and the appropriate SEQ ID NO.

TABLE 1

| SEQ ID NO. | GENE |
|---|---|
| 1 | Sit1 locus |
| 2 | Sit1A |
| 4 | Sit1B |
| 6 | Sit1C |
| 8 | Sit1D |
| 10 | PPI1 locus |
| 11 | Sit2A |
| 13 | Sit2B |
| 15 | Sit2C |
| 17 | Sit2D |
| 19 | Orf1 |
| 21 | Orf2 |
| 23 | Orf3 |
| 25 | Orf4 |
| 27 | Orf5 |
| 29 | Orf6 |
| 31 | Orf7 |
| 33 | Orf8 |
| 35 | Orf9 |
| 37 | Orf10 |
| 39 | Orf11 |
| 41 | Orf12 |
| 43 | Orf13 |
| 45 | Orf14 |
| 47 | MS1 |
| 49 | MS2 |
| 51 | MS3 |
| 53 | MS4 |
| 55 | MS5 |
| 57 | MS6 |
| 59 | MS7 |
| 61 | MS8 |
| 63 | MS9 |
| 65 | MS10 |
| 67 | MS11 |
| 69 | Sit3A |
| 71 | Sit3B |
| 73 | Sit3C |
| 75 | Sit3D |

The invention is now further described by the following Example, with reference to the accompanying figures.

Bacterial Strains, Media and Culture Conditions

A type 3 S. pneumoniae strain, 0100993, isolated from a patient with pneumonia was used for all experiments. S. pneumoniae strains were cultured on Columbia agar supplemented with 5% horse blood, in Todd-Hewitt broth supplemented with 0.5% yeast extract (THY), or using a modified version of RPMI medium at 37° C. and 5% CO₂. RPMlm was made by adding to the tissue culture medium RPMI (type 1640, Gibco) 0.4% bovine serum albumin factor V (BSA, Sigma), 1% vitamin solution and 2 mM glutamine. Medium was cation depleted by adding 2% (THY) or 6% (RPMIm) Chelex (Biorad) to broth medium for 8 (THY) or 24 (RPMI) hours under continuous agitation. THY was autoclaved and the Chelex removed by filter sterilisation before use, and Chelexed RPMI was filter sterilised and used immediately. Chelex-THY and Chelex-RPMIm were supplemented with 100 μM CaCl₂ and 2 mM MgSO₄ before use. When necessary the following supplements were added to medium: chloramphenicol (cm) 4 μg ml⁻¹, erythromycin (ery) 0.4 μg ml⁻¹, 10 to 50 μM FeCl₃, 10 to 50 μM FeSO₄, 1 to 5 μg ml⁻¹ lactoferrin (Sigma), 1 to 5 μg ml⁻¹ ferritin (Sigma), 5 to 10 μM human haemoglobin (Sigma), 1 to 10 μM haemin (Sigma), and 400 μM 2,2′-dipyridyl (Sigma), 25 μM MnSO₄; 25 μM ZnSO₄. Growth of broth cultures was monitored by measuring optical density at 580 nm using a Multiskan Plus MkII plate reader (Titertek) and 200 μl of culture aliquots in 96 well microtitre dishes, or a UV-1201 spectrophotometer and 1 ml cultures in sterile cuvettes. Aliquots of strains grown in THY broth to an OD₅₈₀ of 0.35 to 0.6 were stored in 10% glycerol at −70° C. To minimise Fe contamination, stock solutions were made using MilliQ treated water and stored in disposable plastic ware and cultures were incubated in disposable plastic ware. Plasmids were manipulated in E. coli strain DH5α, grown at 37° C. on Luria-Bertani (LB) medium with appropriate selection (Sambrook et al., 1989).

DNA Isolation and Manipulation

Plasmid DNA was isolated from E. coli using Qiagen Plasmid Kits (Qiagen) using the manufacturer's protocols. Standard protocols were used for cloning, transformation, restriction digests, and ligations of plasmid DNA (Sambrook et al., 1989). Nylon membranes for Southern hybridisations were prepared and probed using ³²P-dCTP labelled probes made using the RediPrime random primer labelling kit (Amersham International Ltd, Bucks, UK) as previously described (Holden et al., 1989). S. pneumoniae chromosomal DNA was isolated using Wizard genomic DNA isolation kits (Promega).

Computer Analysis of Nucleic Acid Sequences

Preliminary S. pneumoniae sequence data was obtained from The Institute for Genomic Research website (http://www.tigr.org) and analysed and manipulated using MacVector (International Biotechnologies, Inc.). BLAST2 searches of available nucleotide and protein databases and of incomplete microbial genomes were performed using the NCBI website (http://www.ncbi.nim.nih.gov/blast/). Dendrograms were constructed using Multalin (http://www.toulouse.inra.fr/multalin.html) and TreeView PPC. Sequence GC content was analysed using Artemis (Genome Research Ltd) and graphs of GC content made with the Window application of the Wisconsin Sequence Analysis Package (Genetics Computer Group).

Construction of Mutant Strains

Plasmids, primers and S. pneumoniae strains constructed and used for this work are shown in Table 2.

TABLE 2

| | |
|---|---|
| NAME | DESCRIPTION/SEQUENCE |

| | |
|---|---|
| strains | |
| 0100993 | serotype III clinical isolate |
| sit1A | 0100993 containing an insetion duplication in Sit1A made with plasmid pPC5: |
| | cm′ |
| Sit2A | 0100993 containing an insertion duplication in Sit2A made with plasmid |
| | pPC12: cm′ |
| sit1A/Sit2A | 0100993 contain an insertion duplication in Sit1A and in Sit2A made with |
| | plasmid pPC12 and pPC25: ery′cm′ |
| PPC4 | 0100993 containing an insertion duplication 265 bp downstream of Sit1D made |
| | with plasmid pPC4: cm′ |
| PPC29 | 0100993 containing an insertion duplication 234 bp downstream of Sit2D made |
| | with plasmid pPC29: cm′ |
| plasmids | |
| pID701 | non-replicating shuttle vector for E. coli and S. pneumoniae derived from |
| | pEVP3: amp′cm′ |
| pACH74 | non-replicating shuttle vector for E. coli and S. pneumoniae: amp′ery′ |
| pPC4 | pID701 with Smt3.3/Smt3.4 PCR product ligated into the Xbal Site: amp′cm′ |
| pPC5 | pID701 with Smt6.1/Smt6.2 PCR product ligated into the Xbal Site (sit1A |
| | disruption vector): amp′cm′ |
| pPC12 | PID7-1 with IRP1.1/IRP1.2 PCR product ligated into the Xbal Site (Sit2A |
| | disruption vector): amp′cm′ |
| pPC25 | pACH74 with Smt6.3/Smt6.4 PCR product ligated into the Kpnl Site (sit1A |
| | disruption vector): amp′ery′ |
| pPC29 | pID701 with IRP1.7/IRP1.8 PCR product ligated into the Xbal Site: amp′cm′ |

| | | |
|---|---|---|
| primers | | |
| Smt6.1 | GCTCTAGACCCACAAGATGCTCTTCG | (SEQ ID NO. 77) |
| Smt6.2 | CGCTCTAGACGCTTGTCGTTAGCGCCACC | (SEQ ID NO. 78) |
| Smt6.3 | GGGGTACCCACAAGATGCTCTTCG | (SEQ ID NO. 79) |
| Smt6.4 | CGGGGTACCGCTTGTCGTTAGCGCCACC | (SEQ ID NO. 80) |
| Smt3.3 | GCTCTAGAAGCTATCGCCGCCCTTGAG | (SEQ ID NO. 81) |
| Smt3.4 | CGCTCTAGAGCAACCTGCGGCTAGTTTCC | (SEQ ID NO. 82) |
| IRP1.1 | GCTCTAGAGTTTTAGATCATGCTTTCGG | (SEQ ID NO. 83) |
| IRP1.2 | CGCTCTAGATTTGTATGCTGCTACAGGAGC | (SEQ ID NO. 84) |
| IRP1.7 | GCTCTAGATTGGGTCAAATGGTTGTGG | (SEQ ID NO. 85) |
| IRP1.8 | CGCTCTAGAACTAGTCGTTGTACTTTC | (SEQ ID NO. 86) |
| ORFB.1 | CACACCGTAATCAAGATC | (SEQ ID NO. 87) |
| ORFB.2 | CTGGTCCGTAATATAGTC | (SEQ ID NO. 88) |
| ORFD.1 | GGTCTATTCGACCACCAG | (SEQ ID NO. 89) |
| ORFD.2 | CTGGTGACCTGCATCAGC | (SEQ ID NO. 90) |
| ORF1.1 | CACGAGTATCTACGTC | (SEQ ID NO. 91) |

S. pneumoniae mutant strains were constructed by insertional duplication mutagenesis. Internal portions of the target genes were amplified by PCR using primers designed from the available genomic sequence, and ligated into pID701 (cm resistance, derived from pEVP3, or pACH74 erythromycin resistant. To make a Sit1A⁻ disruption vector, pPC5, an internal portion of Sit1A from bp 320 to 711 was amplified using primers Smt6.1 and Smt6.2, digested with Xbal and ligated into the Xbal Site of pID701. To make a Sit2A⁻ disruption vector, pPC12, an internal portion of Sit2A from bp 84 to 428 was amplified using primers IRP1.1 and IRP1.2, digested with Xbal and ligated into the Xbal Site of pID701. To make a Sit1A⁻ disruption vector, pPC25, for construction of the double mutant an internal portion of Sit1A from bp 320 to 711 was amplified using primers Smt6.3 and Smt6.4, digested with Kpnl and ligated into the Kpnl Site of pACH74. Vectors designed to insert plasmid DNA 265 bp downstream of the stop codon of Sit1D (plasmid pPC4) and 234 bp downstream of Sit2D (plasmid pPC29) were constructed by ligating PCR products generated by primer pairs Smt3.3/Smt3.4 and IRP1.7/IRP1.8 respectively into the Xbal Site of pID701. The inserts of the disruption vectors were sequenced to confirm that they contained the predicted genomic sequences for their respective PCR primers.

S. pneumoniae strains were transformed using a modified protocol requiring induction of transformation competence with Competence Stimulating Peptide 1 (CSP1) (Håvarstein et al., 1995). 8 ml cultures of S. pneumoniae grown to an OD₅₈₀ between 0.012 and 0.020 in THY broth pH 6.8 were collected by centrifugation at 20000 g at 4° C. and resuspended in 1 ml THY broth pH 8.0 supplemented with 1 mM CaCl₂ and 0.2% BSA. Competence was induced by addition of 400 ng of CSP1 followed by the addition of 10 μg of circular transforming plasmid. The transformation reactions were incubated at 37° C. for 3 hours then plated on selective medium and incubated for 24 to 48 hours at 37° C. in 5% CO₂. Individual mutants were made by transformation of the wild-type S. pneumoniae strain with the appropriate plasmid, and the double Sit1A⁻/Sit2A⁻ strain was constructed by transformation of the Sit2A⁻ strain with pPC25. The identity of the mutations carried by mutant strains was confirmed by PCR and Southern hybridisation. All mutations except pPC29 were stable after two 8 hour growth cycles in THY broth without antibiotics (Sit1A⁻, Sit2A⁻, and pPC4 100% of 100 colonies resistant to chloramphenicol; Sit1A⁻/Sit2A⁻ 100% of 100 colonies resistant to chloramphenicol and 100% to erythromycin; pPC2965% of 100 colonies resistant to chloramphenicol).

Streotonigrin Sensitivity Assays

For bacterial survival streptonigrin assays, stocks of S. pneumoniae strains grown in Chelex-THY broth and stored at −70° C. were defrosted on ice, pelleted by centrifugation at 20000 g at 4° C. and resuspended in THY to which 2.5 μg ml⁻¹ of streptonigrin (Sigma) was then added. The reactions were incubated at 37° C., and aliquots of the reaction cultures taken at 40 and 60 minutes after adding streptonigrin were diluted and plated. Cfu at each time point were expressed as a percentage of the cfu prior to adding streptonigrin and the results presented as a ratio of the wild-type strain's results. To assess sensitivity to streptonigrin discs, S. pneumoniae strain stocks cultured in Chelex-THY broth were plated on RPMlm plates with or without 50 μM FeCl₃ and 400 μM DIP supplementation at a density of several thousand colonies per plate. Antibiotic discs impregnated with 5 μg streptonigrin were placed onto the plates. After incubation for 20 hours at 37° C. in 5% CO₂, the width of the zone of growth inhibition surrounding each disc was measured and confidence intervals for three separate results calculated.

⁵⁵Fe Transport Assays

⁵⁵Fe transport assays were modified from previously described protocols (Bearden and Perry, 1999). Stocks of S. pneumoniae strains cultured in THY broth and stored at −70° C. were defrosted on ice and 5×10⁷cells added to 3 mls of RPMlm. After 1 hour incubation at 37° C. in 5% CO₂, ⁵⁵FeCl₃ (NEN) to a final concentration of 0.2 μCi ml⁻¹ was added. The reactions were incubated for a further 15 or 30 minutes at 37° C., then filtered through 0.45 μM nitrocellulose filters (Millipore), washed with 10 ml RPMI, and allowed to dry. To reduce background radioactivity, the nitrocellulose filters were prefiltered with 40 μM FeCl₃ and ⁵⁵FeCl₃ medium filtered through 0.2 μM membranes (Sartorius) before use. Reaction filters were placed in 10 ml of Optisafe scintillation fluid (Wallac) and counted using the ³H settings of a Beckman LS 1801 scintillation counter (Beckman).

In vivo Studies using Mice Models of S. pneumoniae Infection

Outbred male white mice (strain CD1, Charles Rivers Breeders) weighing from 18 to 22 g were used for all animal experiments except for the Sit1D and Sit2A immunisation experiment. Inocula consisted of appropriately diluted defrosted stocks of S. pneumoniae strains cultured in THY broth and stored at −70° C. Strains being compared by competitive infection were mixed in proportions calculated to result in each strain contributing 50% of the cells in the inoculum. For the pneumonia model, mice were anaesthetised by inhalation of halothane (Zeneca) and inoculated intranasally (IN) with 40 ul of 0.9% saline containing between 5×10⁵ to 5×10⁶ bacteria. For the systemic model, mice were inoculated by intraperitoneal injection with 100 ul of 0.9% saline containing 5×10¹ (for survival curves) or 1×10³ (for competitive infections) bacteria. Three to five mice were inoculated per competitive infection experiment and sacrificed after 24 hours (I P inoculations) or 48 hours (IN inoculations). Target organs were recovered, homogenised in 0.5 ml 0.9% saline and dilutions plated and incubated overnight at 37° C. in 5% CO₂ on non-selective and selective medium. Results for the competitive infections were expressed as competitive indices (CI), defined as the ratio of mutant to wild-type strain recovered from the mice divided by the ratio of mutant to wild type strain in the inoculum (Beuzón et al., 2000). Mice inoculated with a pure inocula of each strain for survival curves were sacrificed when the mice exhibited the following clinical signs of disease; severely ruffled fur, hunched posture, poor mobility, weight loss, and (for IN inoculation only) coughing and tachypnoea. CIs were compared to 1.0 (the predicted CI if there is no difference in virulence between the two strains tested) using Student's t test, and survival curves were compared using the log rank method.

Identification and Sequence Analysis of the Sit1 and Sit2 Genetic loci

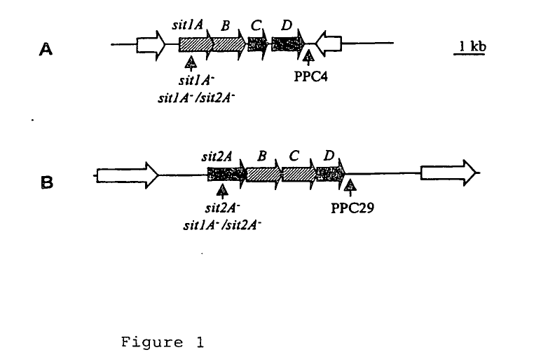

A signature-tagged mutagenesis screen of S. pneumoniae strain in a mouse model of pneumonia identified a strain attenuated in virulence which contains a mutation in a gene (smtA) whose predicted amino acid product has 31% identity and 53% similarity to CeuC, a component of a Campylobacter coli iron uptake ABC transporter (Richardson and Park, 1995). Analysis of the surrounding genome sequence (available at the TIGR website for unfinished microbial genomes, http://www.tigr.org) showed that smtA is the second gene of a four gene locus encoding a likely ABC transporter with high degrees of identity to iron uptake ABC transporters (FIG. 1). This four gene locus was renamed Sit1ABC and D (streptococcal iron transporter 1). Searches of the S. pneumoniae genome using IRP1, an iron regulated Corynebacterium diptheriae lipoprotein, identified a gene, Sit2A, the predicted amino acid sequence of which has 43% identity and 62% similarity to IRP1 (Lee et al., 1997). Analysis of the genome sequence showed that Sit2A is the first gene of a second four gene locus, Sit2ABC and D, with high degrees of identity to iron uptake ABC transporters (FIG. 1). Both the Sit1 and Sit2 loci conform to the reported organisation of loci encoding ABC transporters and contain one gene encoding putative ATPases, one gene encoding putative lipoprotein iron receptors, and two genes encoding putative transmembrane permease proteins (FIG. 1). In both loci all four genes are transcribed in the same direction and either have short intergenic sequences (maximum 135 bp between Sit1B and Sit1C) or overlapping ORFs suggesting they are single transcriptional units. There is a putative 19 bp hairpin loop terminator sequence 72 bp downstream of Sit1D. No hairpin loop terminator sequences were identified downstream of Sit2D.

The Sit1 locus is flanked by an ORF whose derived amino acid sequence has 43% identity to UDP galactose epimerase of Lactococcus lacti (terminates 392 bp upstream of Sit1A, transcribed in the same direction) and a small ORF whose derived amino acid sequence has high degrees of similarity to ORFs of unknown function (starts 230 bp downstream of Sit1D, transcribed in the opposite direction) (FIG. 1). The Sit2 locus is flanked by an ORF whose derived amino acid sequence has 42% identity to a Bacillus subtilis RNA methyltransferase homolog (terminates 1353 bp upstream of Sit2A, transcribed in the same direction), and an ORF whose derived amino acid sequence has 25% identity to a Staphylococcus aureus recombinase (starts 1997 bp downstream of Sit2D, transcribed in the same orientation) (FIG. 1). The degrees of identity and similarity between the derived amino acid sequence of the putative metal-binding receptors of Sit1, Sit1D, and Sit2, Sit2A, is low at 22% and 53%. Sit1D and Sit2A have the highest degree of identities to separate groups of iron compound binding lipoproteins. Sit1D and Sit2A both contain motifs matching the consensus sequence for the lipoprotein signal peptide cleavage site (Sutcliffe and Russell, 1995). The derived amino acid sequences of the putative ATPases, Sit1C and Sit2D, contain motifs commonly found in ATP-binding proteins (Linton and Higgins, 1998).

To assess the role of the Sit loci in iron uptake and virulence, strains carrying mutations of Sit1A and Sit2A were constructed by insertional duplication mutagenesis. Insertions were placed in the first genes of each loci to ensure complete inactivation of operon function. A third mutant containing insertions in both Sit1A and Sit2A was also constructed. To ensure the mutant phenotypes were not due to polar effects of the insertions on genes downstream of the Sit loci, two further mutants, PPC4 and PPC29, were constructed containing insertions 73 and 100 bp downstream of the stop codons of Sit1D and Sit2D respectively (FIG. 1).

Growth of Sit⁻ Mutants on Iron-Deficient Medium

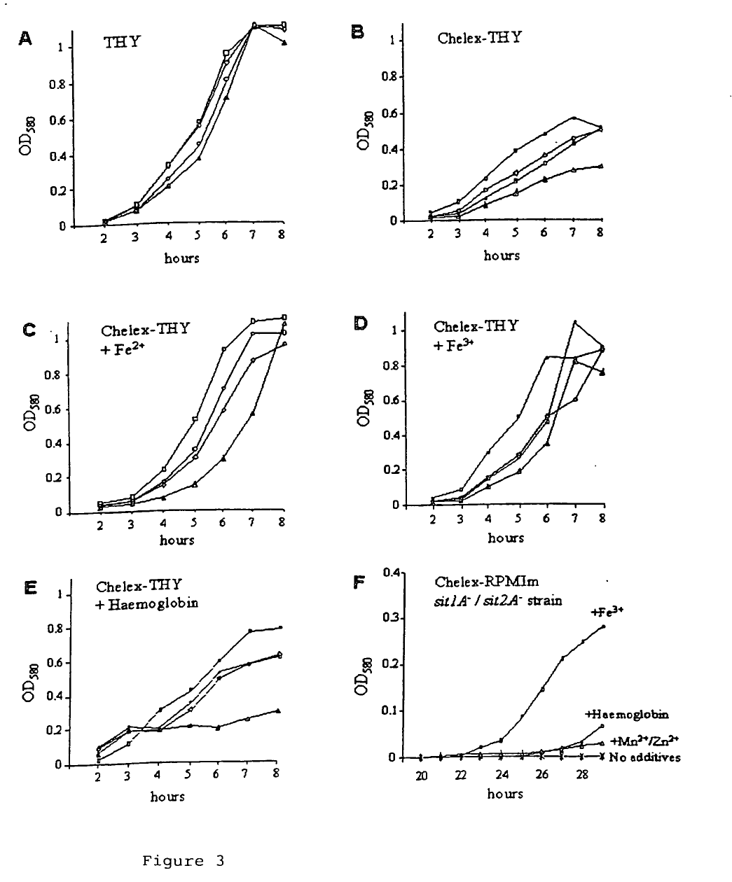

The growth of the Sit mutant strains were compared with the wild-type strain in an undefined complete medium, THY, and in THY which had been treated with Chelex-100 to remove cations (FIG. 3). There were no discernible differences in growth between all the mutant strains and the wild-type strain in THY. Compared to growth in THY all strains had impaired growth in Chelex-THY, and in this medium the double mutant Sit1A⁻/Sit2A⁻ strain had decreased growth compared to wild type and the single Sit⁻ strains (FIG. 3). The wild-type and single Sit1A⁻ and Sit2A⁻ strains' growth defects in chelex-THY were reversed by supplementation with 40 μM ferric or ferrous chloride, partially reversed by supplementation with 10 μM haemoglobin but not by 5 μg ml⁻¹ of lactoferrin or 5 pg ml⁻¹ of ferritin. Growth of the double mutant Sit1A⁻/Sit2A⁻ strain in chelex-THY was restored to levels similar to that of the wild-type strain by the addition of ferric or ferrous chloride, suggesting that this strain's growth defect compared to the wild-type strain in chelex-THY is due to iron depletion. However, supplementing Chelex-THY with 10 μM haemoglobin did not restore growth of the Sit1A⁻/Sit2A⁻ strain, indicating that this strain is unable to use haemoglobin as an iron source.

These findings were confirmed by investigating the growth of the Sit⁻ strains in a defined medium with no added iron, RPMlm. Growth of the wild-type strain in Chelex-RPMlm was delayed and reduced compared to growth in Chelex-THY but could be markedly improved by the addition of 5 μM haemoglobin or haemin. Growth of the wild-type strain in Chelex-RPMlm was partially inhibited by ferrous chloride, ferric citrate and ferric chloride, possibly due to competitive inhibition of the available metal ion transporters by free iron resulting in reduced uptake of other cations. The double Sit1A⁻/Sit2A⁻ strain was unable to grow in Chelex-RPMlm without supplementing the medium with 10 μM ferric or ferrous chloride. Supplementation with 5 μm haemoglobin or haemin, or 25 μM Mn and Zn had a minimal effect on growth. These results confirm that the double Sit1A⁻/Sit2A⁻ strain is unable to use haemoglobin as an exogenous source of iron when growing in iron restricted medium. Hence, one substrate of the Sit1 and Sit2 loci iron transporters is likely to be haemoglobin and loss of function of either Sit1 or Sit2 can be compensated for by the other iron transporter.

Sit1A⁻ and Sit2A⁻ Mutants have Decreased Sensitivity to Streptonigrin

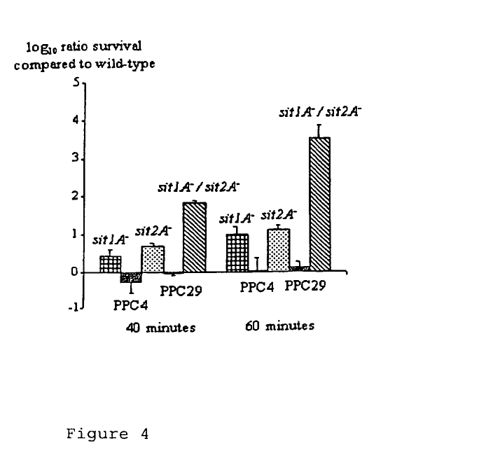

The bacteriocidal effects of the antibiotic streptonigrin requires intracellular iron (Yeowell and White, 1982). Hence, decreased sensitivity to streptonigrin is evidence of lower intracellular levels of iron and this property has been exploited to identify iron transporter mutations (Braun et al., 1983; Pope et al., 1996). The streptonigrin sensitivity of the Sit⁻ strains were assessed by comparing the proportional survival of mutant to wild-type strains when incubated with streptonigrin and by measuring the zone of growth inhibition around a streptonigrin disc when plated on RPMIm medium (FIG. 4). Both methods showed that the Sit⁻ strains were less sensitive to streptonigrin than the wild-type strain. Approximately 10-fold more Sit1A⁻ and Sit2A⁻ cells and a 1000-fold more Sit1A⁻/Sit2A cells survived 60 minutes incubation with streptonigrin than wild type cells (FIG. 4), and the zone of growth inhibition surrounding a streptonigrin disc was smaller for the Sit strains than for the wild-type strain. Supplementation with the metal chelator 2,2′-dipyridyl (DIP) accentuated differences in streptonigrin sensitivity between the Sit⁻ strains and the wild-type strain. Strikingly, in the presence of 400 μM DIP the Sit1A⁻/Sit2A⁻ double mutant was completely resistant to 5 ug streptonigrin discs. The addition of 25 μM FeCl₃, but not 25 μM MnSO₄, to plates supplemented with DIP restored streptonigrin sensitivity, confirming that the efficacy of streptonigrin is iron-dependent. These results show the Sit⁻ cells contain less iron than wild-type bacteria and provide indirect evidence that the Sit loci encode iron transporters. The Sit2A⁻ strain was less sensitive to streptonigrin than the Sit1A⁻ strain, suggesting it may be the dominant iron transporter of the two. Disrupting both Sit loci has a clear additive effect, resulting in a strain highly resistant to streptonigrin. Mutant strains containing insertions immediately downstream of the Sit loci, pPC4 and pPC29, had streptonigrin sensitivities similar to the wild-type strain, confirming that the loss of streptonigrin sensitivity of the Sit strains is due to the mutations in the Sit loci. Although pPC29 does not contain a stable mutation, over 50% of the culture tested in the streptonigrin sensitivity experiments remained chloramphenicol resistant. Hence, if pPC29 is less sensitive to streptonigrin then a partial phenotype would have been identified.

⁵⁵FeCl₃ Uptake by Sit⁻ Mutant Strains

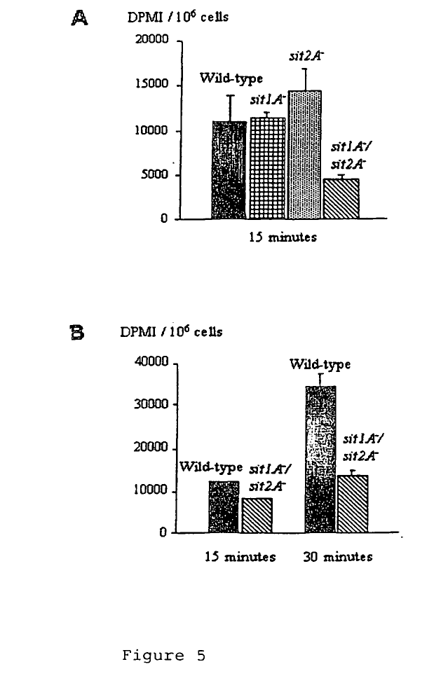

Direct evidence for a role in iron uptake of the Sit loci was obtained by measuring the uptake of the radioactive isotope ⁵⁵FeCl₃ by the mutant and wild-type strains (FIG. 5). After 15 minutes incubation with ⁵⁵FeCl₃ no differences were detected between the wild-type and the single Sit1A⁻ and Sit2A⁻ strains. However, the ⁵⁵FeCl₃ level was significantly lower for the Sit1A⁻Sit2A⁻ strain. To show that the lower level of ⁵⁵FeCl₃for the Sit1A⁻/Sit2A⁻ strain at 15 minutes was due to a reduced rate of iron uptake, the levels of ⁵⁵FeCl₃ for the wild-type and Sit1A⁻/Sit2A⁻ strains were compared after 15 and 30 minutes incubation with ⁵⁵FeCl₃. Between 15 and 30 minutes the ⁵⁵FeCl₃ content of the wild-type strain increased by 280% whereas the ⁵⁵FeCl₃ content of the Sit1A⁻/Sit2A⁻ strain increased by 160%, confirming that the Sitl and Sit2 loci function as iron transporters.

Virulence of Sit1A⁻ and Sit2A⁻ Strains in a mouse model of Pneumonia and Systemic Infection

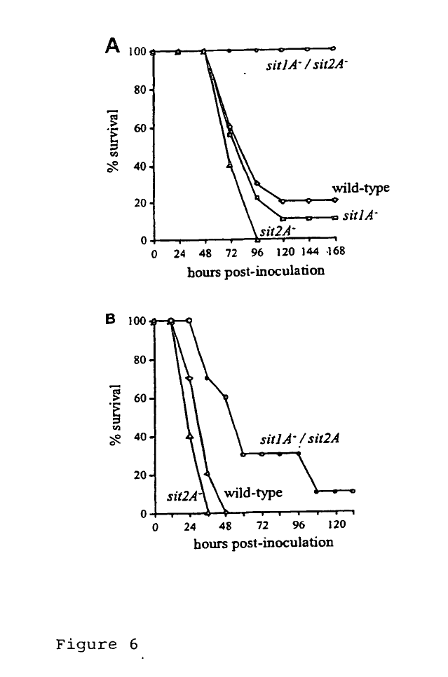

The effect on virulence of mutations in either Sit1A⁻ or Sit2A⁻ or both genes was investigated in mouse models of pulmonary (intranasal inoculation, IN) and systemic infection (intraperitoneal inoculation, IP) (FIG. 6). Subtle differences in virulence were assessed using competitive infections of the mutant strains versus the wild-type strain and the ability of the strains to cause fatal disease was assessed using survival curves. The results for the competitive infections are expressed as the ratio of mutant to wild-type colonies recovered from the target organ divided by the ratio of mutant to wild-type strains in the inoculum (the competitive index, CI) (Beuzón et al., 2000).

In competitive infections against the wild-type strain the Sit1A⁻ strain was mildly attenuated in pulmonary infection (lungs CI=0.67, SD0.07; spleen CI 0.4, SD 0.34), but was not attenuated in the systemic infection model (spleen CI 1.13). The Sit2A⁻ strain was clearly attenuated in both the pulmonary infection model (lung CI 0.13, SD 0.14; spleen CI 0.14, SD 0.15) and in the systemic infection model (spleen CI 0.32, SD 0.11). Hence, the Sit loci seem to have different roles during infection, with Sit2 being of greater importance for both pulmonary and systemic infection. The mutant strain containing an insertion immediately downstream of the Sit1 locus, pPC4, was not reduced in virulence, confirming the reduced virulence of the Sit1A⁻ strain was not due to a polar effect. Due to its relative instability, the mutant strain containing an insertion immediately downstream of the Sit2 locus, pPC29, was not tested in vivo. The double Sit1A⁻/Sit2A⁻ strain was considerably reduced in virulence compared to the wild-type strain in both the pulmonary and systemic models of infection. No Sit1A⁻/Sit2A⁻ colonies were recovered from the spleen 24 hours after IP inoculation in a mixed inoculum with the wild-type strain, and approximately 10³ fewer Sit1A⁻/Sit2A⁻ colonies were recovered from the lungs than wild-type colonies after IN inoculation (1.3×10⁴v.6.8×10⁷ respectively, n=3). These results suggest that mutation of both Sit1A⁻ and Sit2A⁻ has a synergistic effect on the virulence of S. pneumoniae. To confirm this finding, the Sit1A⁻/Sit2A⁻ strain was compared to the Sit2A⁻ strain in a pulmonary competitive infection. If the mutations in the Sit1 and Sit2 loci had effects on unconnected virulence functions, the consequences on the CI of the Sit2A⁻ mutation would affect both strains in the inoculum equally. Hence, the CI for the Sit1A⁻/Sit2A⁻ strain versus the Sit2A⁻ strain would be similar to the CI of a Sit1A⁻ strain versus wild-type (CI=0.67). However, the CI was less than 0.001 demonstrating that dual mutation of Sit1 and Sit2 has a synergistic effect on S. pneumoniae virulence.

No differences were found in the mortality of mice inoculated with the wild type or the single Sit1A⁻ and Sit2A⁻ strains either in pulmonary (inoculum 5×10⁶ cfu, 80 to 100% mortality after 5 days) or in systemic infection (inoculum 50 cfu, 100% mortality after 48 hours) (FIG. 6). However the double mutant Sit1A⁻/Sit2A⁻ strain was highly attenuated in the pulmonary infection model. After a transient illness with mild pilo-erection and decrease in mobility during the first 36 hours post-inoculation, all mice inoculated with the Sit1A⁻/Sit2A⁻ strain recovered and survived the duration of the experiment. In contrast, the mortality of mice due to systemic infection with either single mutant (Sit1A⁻ or Sit2A⁻) strain was 90%. However the time of death was significantly delayed compared to the wild-type strain (p=0.002, log rank test).

Sit2 is Encoded on a Pathogenicity Island

The closest homolog of the ORF immediately downstream of the Sit2 locus (ORF 1) is a putative recombinase carried by the S. aureus mec mobile genetic element. mec is an ‘antibiotic resistance island’ which confers methicillin resistance, and the recombinase may catalyse recombination of mec into S. aureus chromosomal DNA (Ito et al., 1999). In addition, analysis of the genome sequence showed that the Sit2 locus has a lower GC content than the upstream DNA sequence, with a striking drop in GC content occurring within the C terminus of the ORF immediately upstream of the Sit2 locus (ORF B). These findings stimulated investigation for further evidence that Sit2 maybe part of a PI using the available genome sequence from a serotype 4 S. pneumoniae strain.

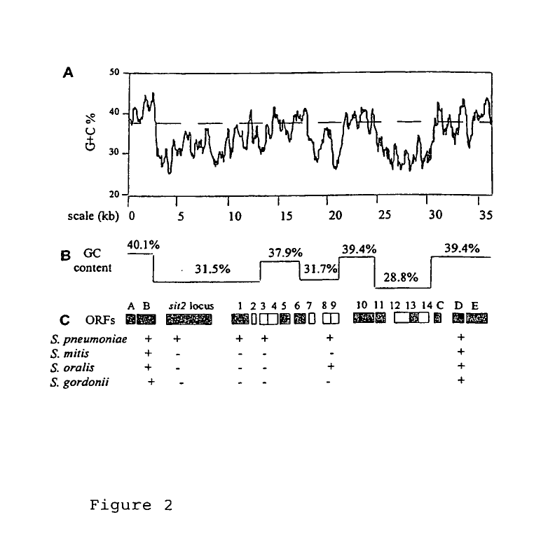

The GC content of 250,000 bp of S. pneumoniae chromosomal DNA on the contig containing Sit1 was 38.9%, similar to the estimate calculated by chemical methods (38.5%, Hardie, 1986). Analysis of the GC content of consecutive 800 bp segments of 41.6 kb of DNA including the Sit2 locus identified an area of approximately 27,000 bp with a mean GC content of 32.6%, nearly 7% lower than the surrounding sequence (p<0.001, FIG. 2). This area was termed PPI1 (Pneumococcal Pathogenicity Island 1). The boundaries of PPI1 are marked by sharp decreases in GC content (FIG. 2) and were defined to within 50 bp by GC content analysis of 200 bp lengths of DNA overlapping by 10 bp. The left-hand boundary lies within the C terminus of the first ORF 5′ to the Sit2 loci which encodes a likely RNA methyltransferase (FIG. 2). The right-hand boundary of PPI1 lies between an ORF with no close homologs in the databases and a probable transposase (FIG. 2). Visual analysis of the DNA sequence around the PPI1 boundaries did not identify inverted or direct repeats. Two areas within PPI1 of approximately 4000 and 3200 bp length have a GC content approaching that of the S. pneumoniae chromosome (FIG. 2). The putative transposase immediately downstream of the 3′ end of PPI1 belongs to the insertion sequence family IS605, which includes the IS200 transposons of Gram-negative pathogens (Mahillon and Chandler, 1998). IS605 transposons have been reported in S. pneumoniae (Oggioni and Claverys, 1999) and are characterised by a low frequency of transposition and hairpin loops 5′ to the transposase, but do not contain terminal inverse repeats (IR) (Beuzon and Casadesus, 1997; Mahillon and Chandler, 1998). In keeping with these data we identified a hairpin loop 159 bp 5′ to the transposase. In addition to being flanked by a transposase, PPI1 contains two ORFs associated with mobile genetic elements, the recombinase described above and a relaxase (ORF11). In contrast to the Sit2 locus, chromosomal DNA around the Sit1 locus has a mean GC content of 40.1% with no regions whose GC content significantly varies from the mean for S. pneumoniae.

In addition to the Sit2 locus within PPI1 there are 14 ORFs whose predicted amino acid products are longer than 100 residues (FIG. 2). Incomplete genome sequences are available for three non-S. pneumoniae streptococcal species (Streptococcus pyogenes, Streptococcus mutans and Streptococcus equli) on the world-wide web (http://www.ncbi.nim.nih.gov/blast/), allowing investigation of the distribution of the ORFs present within and adjacent to PPI1 amongst streptococci (FIG. 2). The five ORFs flanking PPI1 have at least 59% identity to ORFs from two or more non-S. pneumoniae streptococcal species, whereas 8 of the 14 ORFs within PPI1 have no identity to ORFs from non-S. pneumoniae streptococcal species. The remaining 6 ORFs have similar levels of identity to ORFs from non-streptococcal species and streptococcal species, varying from 22% to 42%. Furthermore, these ORFs include parts of mobile elements (e.g. ORF1 is a recombinase, ORF10 is a relaxase) or belong to families of proteins which have multiple representatives within a given genome (ORF13 is a likely ATPase and ORF11 is a possible transcription factor). Sit2A has high degrees of similarity only to ORFs from non-streptococcal species whereas Sit1D has high degrees of identity to an ORF from Streptococcus mutans (35 % identity and 52% similarity over 332 residues).

The results of the BLAST searches suggested that the ORFs flanking PPI1 are present in distantly related streptococcal species whereas Sit2 and the ORFs within PPI1 are not. The distribution of the Sit loci and the ORFs within and surrounding PPI1 within streptococcal species closely related to S. pneumoniae was investigated by Southern analysis using internal fragments of the Sit genes and PPI1 ORFs as probes under non-stringent conditions. The Sit1D probe hybridised to genomic DNA fragments from S. mitis, S. sanguis, S. oralis and S. milleri, whereas the Sit2A probe only hybridised to S. pneumoniae DNA. Hence, Sit1 is widely distributed amongst Streptococcus species closely related to S. pneumoniae whereas Sit2 is restricted to S. pneumoniae. The presence of the Sit loci in different S. pneumoniae strains was investigated using PCR with primers specific for internal fragments of Sit1A and Sit2A (Smt6.½ and IRP1.½). Both genes were present in all S. pneumoniae strains investigated. The results of Southern analysis of different streptococcal species probed under with internal fragments of ORFs within and adjacent to PPI1 are represented in FIG. 2. To summarise, hybridisation signals were obtained from all viridans streptococci for ORF B and D which flank the 5′ end of PPI1 and the transposase at the 3′ end of PPI1 respectively. However, ORFs within PPI1 usually gave no hybridisation signals except from S. pneumoniae, confirming that PPI1 is only present in S. pneumoniae and is not present in the streptococcal species most closely related to S. pneumoniae.

The experiments disclosed herein also revealed a third iron transport system (Sit3) which contained four gene sequences (SitA, B, C and D). The gene sequences are identified in the accompanying sequence listing.

Sit1D and Sit2A Immunisation Experiment

This experiment illustrates the effectiveness of a vaccine comprising a combination of Sit1D and Sit2A proteins.

BALBc were mice given 10 ug of protein (expressed in pQE30 expression vectors in E. coli and purified using the His-tag) via IP, on three occasions separated by 7-10 days, and then challenged 2 weeks after the last immunisation with 10,000 S. pneumoniae cells inoculated IP.

Alum was used as a negative control, and the non-toxic pneumolysin variant, termed Pdb, was used as a positive control (known to be protective). The other proteins were Sit1D, Sit2A, Sit1D combined with Sit2A, Sit1D combined with Pdb, and Sit2A combined with Pbd.

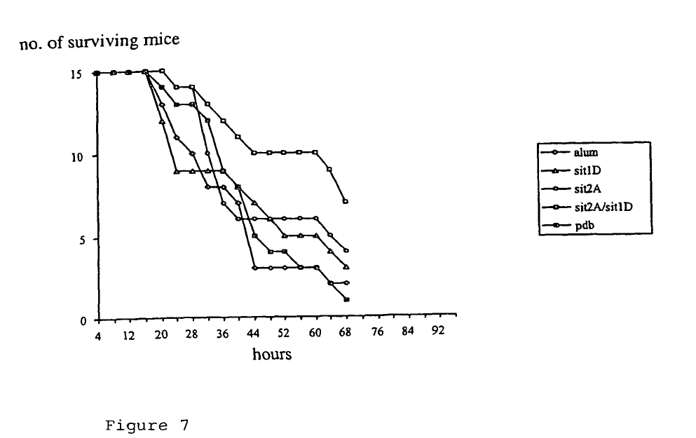

Essentially, both Sit1D and Sit2A are as protective as Pdb, and the combination of Sit1D and Sit2A is very protective (80% long term survivors compared to 0% in the alum group). Combinations of Pdb and either Sit1D or Sit2A had no additional protective benefit over the individual proteins (FIG. 7).

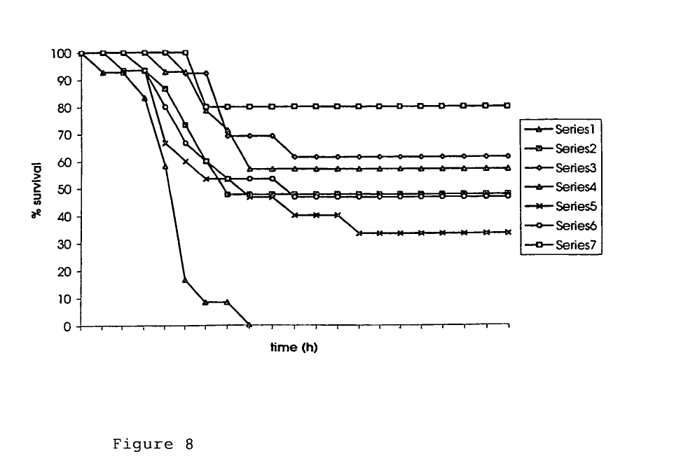

To show that the protective effect is antibody-mediated, the serum from immunised mice was given IP to another group of naive mice, and then these mice were challenged with 3000 bacteria. The results showed a clear benefit for the group receiving the combined Sit1D/Sit2A antisera. The clear positive result with the Sit1D/Sit2A antisera confirms that the protective effect of immunisation with Sit1D and Sit2A is a serum, i.e. antibody, dependent phenomena (FIG. 8).

References

Bearden, S. W., and Perry, R. D. (1999) The Yfe system of Yersinia pestis transports iron and manganese and is required for full virulence of plague. Mol Microbiol 32: 403-414.

Beuzón, C. R., and Casadesús, J. (1997) Conserved structure of IS200 elements in Salmonella typhimurium. Nucleic Acids Res 25: 1355-1361.

Beuzón, C. R., Méresse, S., Unsworth, K. E., Ruiz-Albert, J., Garvis, S., Waterman, S. R., Ryder, T. A., Boucrot, E., and Holden, D. W. (2000) Salmonella typhimurium maintains the integrity of its intracellular vacuole through the action of SifA. EMBO J 19: 3235-3249.

Blanc-Potard, A. B., and Groisman, E. A. (1997) The Salmonella selC locus contains a pathogenicity island mediating intramacrophage survival. EMBO J 16: 5376-85.

Blanc-Potard A. B., Solomon, F., and Groisman, E. A. (1997) The SPI-3 pathogenicity island of Salmonella enterica. J Bacteriol 181: 998-1004.

Braun, V., Gross, R., Koster W., and Zimmermann, L. (1983) Plasmid and chromosomal mutants in the iron (III)-aerobactin transport system of Escherichia coli. Use of streptonigrin for selection. Mol Gen Genet 192: 131-139.

Braun et al., 1997 Clostridia (don't have the paper for this one—use medline)

Carniel, E., Guilvout, I., and Prentice, M. (1996) Characterization of a large chromosomal “high pathogenicity island” in biotype 1 B Yersinia enterocolitica. J Bacteriol 178: 6743-6751.

Claverys, J. P., Prudhomme, M., Mortier-Barrière, I., and Martin, B. (2000) Adaptation to the environment: Streptococcus pneumoniae, a paradigm for recombination-mediated genetic plasticity. Mol Microbiol 35: 251-259.

Cornelissen, C. N., and Sparling, P. F. (1994) Iron piracy: acquisition of transferrin-bound iron by bacterial pathogens. Mol Microbiol 14: 843-850.

Fetherstone, J. D., Bertolino, V. J., and Perry, R. D. (1999) YbtP and YbtQ: two ABC transporters required for iron uptake in Yersinia pestis. Mol Microbiol 32: 289-299.

Fetherstone, J. D., and Perry, R. D. (1994) The pigmentation locus of Yersinia pestis KIM6+ is flanked by an insertion sequence and includes the structural genes for pesticin sensitivity and HMWP2. Mol Microbiol 13: 697-708.

Hacker, J., Blum-Oehler, G., Mühldorfer, I., and Tschäpe, H. (1997) Pathogenicity islands of virulent bacteria: structure, function and impact on microbial evolution. Mol Microbiol 23:1089-1097.

Hardie, J. M. (1986) Genus Streptococcus. In Krieg, N. R., and Holt, J. G. (ed.) Bergey's Manual of Systematic Bacteriology. Williams & Wilkins, Baltimore, pp. 1043-1071.

Håvarstein, L. S., Coomaraswami, G., and Morrison, D. A. (1995) An unmodified heptadecapeptide pheromone induces competence for genetic transformation of Streptococcus pneumoniae. Proc Natl Acad Sci USA 92: 11140-11144.

Hensel et al., (1995) Science 269:400-403.

Holden, D. W., Kronstad, J. W., and Leong, S. A. (1989) Mutation in a heat-regulated hsp70 of Ustilago maydis. EMBO J 8: 1927-1934.

Ito, I., Katayama, Y., and Hiramatsu, K. (1999) Cloning and nucleotide sequence determination of the entire mec DNA of pre-methicillin-resistant Staphylococcus aureus N315. Antimicrob Agents Chemother 43: 1449-1458.

Janakiraman, A., and Slauch, J. M. (2000) The putative iron transport system SitABCD encoded on SPI1 is required for full virulence of Salmonella typhimurium. Mol Microbiol 35: 1146-1155.

LaForce, F. M., Boose, D. S., and Ellison, R. T. (1986) Effect of aerosolised Escherichia coli and Staphylococcus aureus on iron and iron-binding proteins in lung lavage. J Infect Dis 154: 959-965.

Lee, J. H., Wang, T., Ault, K., Liu, J., Schmitt, M. P., and Holmes, R. K. (1997) Identification and characterisation of three new promoter/operators from Corynebacterium diphtheriae that are regulated by the diphtheria toxin repressor (DtxR) and iron. Infect Immun 65: 4273-4280.

Lindsay, J. A., Ruzin, A., Ross, H. R., Kurepina, N., and Novik, R. (1998) The gene for toxic shock is carried by a family of mobile pathogenicity islands in Staphylococus aureus. Mol Microbiol 29: 527-543.

Linton, K. J., and Higgins, C. F. (1998) The Escherichia coli ATP-binding cassette (ABC) proteins. Mol Microbiol 28: 5-13.

Mahillon, J., and Chandler, M. (1998) Insertion sequences. Microbiol Mol Biol Rev 62: 725-774.

Ochman, H., Lawrence, J. G., and Groisman, E. A. (2000) Lateral gene transfer and the nature of bacterial innovation. Nature 405: 299-304.

Oggioni, M. R., and Claverys, J. P. (1999) Repeated extragenic sequences in prokaryotic genomes: a proposal for the origin and dynamics of the RUP element in Streptococcus pneumoniae. Microbiology 145: 2647-2653. 3028-3033.

Pope, C. D., O'Connell, W., and Cianciotto, N. P. (1996) Legionella pneumophila mutants that are defective for iron acquisition and intracellular infection. Infect immun 64: 629-636. of Listeria monocytogenes pathogenesis. Infect Immun 60: 1263-1267.

Richardson, P. T., and Park, S. F. (1995) Enterochelin acquisition in Campylobacter coli: characterisation of components of a binding-protein-dependent transport system. Microbiology 141: 3181-3191.

Sambrook, J., Fritsch, E. F., and Maniatis, T. (1989) Molecular cloning, a laboratory manual, 2nd edition, Cold Spring Harbor Press.

Schryvers, A. B., and Stojiljkovic, I. (1999) Iron acquisition systems in the pathogenic Neisseria. Mol Microbiol 32: 1117-1123.

Shea, J. E., Hensel, M., Gleeson, C., and Holden, D. W. (1996) Identification of a virulence locus encoding a second type III secretion system in Salmonella typhimurium. Proc Natl Acad Sci USA 93: 2593-2597.

Sutcliffe, I., and Russell, R. R. B. (1995) Lipoproteins of Gram-positive bacteria. J Bacteriol 177: 1123-1128.

Tai, S. S., Lee, C., and Winter, R. E. (1993) Hemin utilization is related to virulence of Streptococcus pneumoniae. Infect Immun 61: 5401-5405.

Tai, S. S., Wang, T. R ., and Lee, C. (1997) Characterisation of hemin binding activity of Streptococcus pneumoniae. Infect Immun 65: 1083-1087.

Takase, H., Nitanai, H., Hoshino, K., and Otani, T. (2000) Impact of siderophore production on Pseudomonas aeruginosa infections in immunosuppressed mice. Infect Immun 68: 1834-1839.

Viswanathan, V. K., Edelstein, P. H., Pope, C. D., and Cianciotto, N. P. (2000) The Legionella pneumophila iraAB locus is required for iron assimilation, intracellular infection, and virulence. Infect Immun 68: 1069-1079.

Wandersman, C., and Stojiljkovic, I. (2000) Bacterial heme sources: the role of heme, hemoprotein receptors and hemophores. Curr Opin Microbiol 3: 215-220.

Wooldridge, K. G., and Williams, P. H. (1993) Iron uptake mechanisms of pathogenic bacteria. FEMS Microbiol Rev 12: 325-348.

Yeowell, H. N., and White, J. R. (1982) Iron requirement in the bacteriocidal mechanism of streptonigrin. Antimicrob agents Chemother 22: 961-968.

1. A peptide encoded by any of the gene sequences identified herein as Sit1 A, B, C, Sit2 B, C or D, Sit3 A, B, D or D, ORF 1 to 14, and MS 1 to 11, or a functional fragment thereof, for therapeutic or diagnostic use.

2. A peptide encoded within the pneumococcal pathogenicity island 1, identified herein as PPI1.

3. A peptide according to claim 2, for therapeutic or diagnostic use.

4. A polynucleotide encoding a peptide according to any of claims 1 to 3, for therapeutic or diagnostic use.

5. A polynucleotide according to claim 4, having at least 40 nucleotides.

6. A polynucleotide according to claim 5, having at least 80 nucleotides.

7. An attenuated microorganism comprising a mutation that disrupts expression of any of the gene sequences identified herein as Sit1 A, B, C or D, Sit2 A, B, C or D, Sit3 A, B, C or D, ORF 1 to 14, and MS 1 to 11.

8. A microorganism according to claim 7, wherein the gene is any of the Sit1, Sit2 or Sit3 genes.

9. A microorganism according to claim 7 or claim 8, wherein the mutation is a deletion mutation.

10. A microorganism according to any of claims 7 to 9, comprising a further attenuating mutation in a second gene.

11. A microorganism according to claim 10, wherein the further mutation is an auxotrophic mutation.

12. A microorganism according to any of claims 7 to 11, genetically modified to express a heterologous antigen.

13. A construct comprising a promoter naturally associated with a gene according to claim 1, and a heterologous gene.

14. A vaccine comprising a microorganism according to any of claims 7 to 11.

15. A vaccine comprising a peptide according to claim 1, or a polynucleotide according to claim 4.

16. A vaccine comprising at least two peptides according to claim 1.

17. A vaccine comprising a peptide encoded by Sit1D and a peptide encoded by Sit2A, or a functional fragment thereof capable of elliciting an immune response.

18. Use of a product according to any of claims 1 to 12, in a screening assay for the identification of an antimicrobial drug.

19. Use of a product according to any of claims 1 to 12, in a diagnostic assay for the detection of a streptococcal microorganism.

20. Use of a peptide encoded by the Sit2A gene or Sit1D gene, in a screening assay for the identification of an antimicrobial drug.

21. Use of a peptide encoded by the Sit2A gene or Sit1D gene, in a diagnostic assay for the detection of a streptococcal microorganism.

22. Use of a product according to any of claims 1 to 12, for the manufacture of a medicament for the treatment or prevention of a condition associated with infection by S. pneumoniae or other Gram-positive bacteria.

23. Use according to claim 22, wherein the treatment is veterinary treatment.

24. An antibody, raised against any of the products of claims 1 to 6.
